SEQUENCE LISTING (2 sequences)

SEQ ID NO: 1 - DNA, 16 bp, artificial
  nucleic acid molecule
cccagcaaacaagcct

SEQ ID NO: 2 - DNA, 10 bp, artificial
  nucleic acid molecule
cagcaacaag

(19) United States
(12) Patent Application Publication
(10) Pub. No.: US 2022/0054524 A1
(43) Pub. Date: Feb. 24, 2022

(54) NEW CONJUGATED NUCLEIC ACID MOLECULES AND THEIR USES

(71) Applicant: ONXEO, PARIS (FR)

(72) Inventors: BRIAN SPROAT, BOOISCHOT (BE); CHRISTELLE ZANDANEL, FONTENAY-AUX-ROSES (FR); FRANÇOISE BONO, TOULOUSE (FR); ALEXANDRE SIMON, PARIS (FR)

(21) Appl. No.: 17/414,342

(22) PCT Filed: Dec. 20, 2019
(86) PCT No.: PCT/EP2019/086672

§ 371 Date: Jun. 16, 2021

(30) Foreign Application Priority Data
Dec. 21, 2018 (EP) 18306826.1
Dec. 21, 2018 (EP) 18306829.5
Oct. 11, 2019 (EP) 19202834.8
Oct. 11, 2019 (EP) 19202837.1

(51) Int. Cl. A61K 31/7088; C12N 15/113

(52) U.S. Cl. CPC A61K 31/7088; C12N 15/113; C12N 2310/315; C12N 2310/531; C12N 2310/344; C12N 2310/345; C12N 2310/322

(57) ABSTRACT

The present invention relates to new nucleic acid molecules of therapeutic interest, in particular for use in the treatment of cancer.

### FIELD OF THE INVENTION

The present invention relates to the field of medicine, in particular of oncology.

### BACKGROUND OF THE INVENTION

DNA-damage response (DDR) detects DNA lesions and promotes their repair. The wide diversity of DNA-lesion types necessitates multiple, largely distinct DNA-repair mechanisms such as mismatch repair (MMR), base-excision repair (BER), nucleotide excision repair (NER), single-strand break repair (SSB) and double-strand break repair (DSB). For example, the polyadenyl-ribose polymerase (PARP) is involved essentially in repairing SSBs while two principal mechanisms are used for repairing DSBs in DNA: non-homologous end-joining (NHEJ) and homologous recombination (HR). In NHEJ, DSBs are recognized by the Ku proteins that then binds and activates the protein kinase DNA-PKcs, leading to recruitment and activation of end-processing enzymes. It has been demonstrated that the ability of cancer cells to repair therapeutically induced DNA damage impacts therapeutic efficacy.

This has led to targeting DNA repair pathways and proteins to develop anti-cancer agents that will increase sensitivity to traditional genotoxic treatments (chemotherapeutics, radiotherapy). Synthetic lethal approaches to cancer therapy have provided novel mechanisms to specifically target cancer cells while sparing non-cancer cells and thereby reducing toxicity associated with treatment.

Amongst these synthetic lethal approaches, Dbait molecules are nucleic acid molecules that mimic double-stranded DNA lesions. They act as a bait for DNA damage signaling enzymes, PARP and DNA-PK, inducing a “false” DNA damage signal and ultimately inhibiting recruitment at the damage site of many proteins involved in DSB and SSB pathways.

Dbait molecules have been extensively described in PCT patent applications WO2005/040378, WO2008/034866 WO2008/084087 and WO2017/013237. Dbait molecules may be defined by a number of characteristics necessary for their therapeutic activity, such as their minimal length which may be variable, as long as it is sufficient to allow appropriate binding of Ku protein complex comprising Ku and DNA-PKcs proteins. It has thus been showed that the length of Dbait molecules must be greater than 20 bp, preferably about 32 bp, to ensure binding to such a Ku complex and enabling DNA-PKcs activation.

Potential predictive biomarkers for treatment with such Dbait molecules were characterized. Sensitivity to Dbait molecules was indeed associated with a high spontaneous frequency of cells with micronuclei (MN) as described in PCT patent application WO2018/162439. A high basal level of MN was proposed as a predictive biomarker for treatment with Dbait molecules consecutive to a validation in 43 solid tumor cell lines from various tissues and 16 models of cell- and patient-derived xenografts.

Moreover, it has been recently proposed that micronuclei (MN) would provide a key platform as part of DNA damage-induced immune response (Gekara J Cell Biol. 2017 Oct. 2; 216(10):2999-3001). Recent studies demonstrate a role for MN formation in DNA damage-induced immune activation. Interestingly, a cytosolic DNA sensing pathway has indeed emerged as the major link between DNA damage and innate immunity. DNA normally resides in the nucleus and mitochondria; hence, its presence in the cytoplasm serves as a danger-associated molecular pattern (DAMP) to trigger immune responses. Cyclic guanosine monophosphate (GMP)-adenosine monophosphate (AMP) synthase (cGAS) is the sensor that detects DNA as a DAMP and induces type I IFNs and other cytokines. DNA binds to cGAS in a sequence-independent manner; this binding induces a conformational change of the catalytic center of cGAS such that this enzyme can convert guanosine triphosphate (GTP) and ATP into the second messenger cyclic GMP-AMP (cGAMP). This cGAMP molecule is an endogenous high-affinity ligand for the adaptor protein Stimulator of IFN Gene STING. Activation of the STING pathway may then include, for example, stimulation of inflammatory cytokines, IP-10 (also known as CXCL10), and CCL5 or receptors NGK2 and PD-L1.

Recent evidence indicates involvement of the STING (stimulator of interferon genes) pathway in the induction of antitumor immune response. Therefore, STING agonists are now being extensively developed as a new class of cancer therapeutics. It has been shown that activation of the STING-dependent pathway in cancer cells can result in tumor infiltration with immune cells and modulation of the anticancer immune response.

STING is an endoplasmic reticulum adaptor that facilitates innate immune signaling (a rapid nonspecific immune response that fights against environmental insults including, but not limited to, pathogens such as bacteria or viruses). It was reported that STING is able to activate NF-kB, STAT6, and IRF3 transcription pathways to induce expression of type I interferon (e.g., IFN-α and IFN-β) and exert a potent anti-viral state following expression. However, STING agonists developed so far are able to activate the STING pathway in all cell types and could trigger dramatic side effects linked to their activation in dendritic cells. In consequence, STING agonists are locally administrated.

Accordingly, there is a real need to find a way to specifically activate the STING pathway in tumor cells.

Accordingly, there remains a need for therapies for cancer treatment, especially drugs which rely on several mechanisms, especially DNA repair pathways and STING pathway activators, and for drugs that may help checkpoint inhibitors to work in more patients and across a wider range of cancers.

Cancer cells have a unique energy metabolism for sustaining rapid proliferation. The preference for anaerobic glycolysis under normal oxygen conditions is a unique trait of cancer metabolism and is designated as the Warburg effect. Enhanced glycolysis also supports the generation of nucleotides, amino acids, lipids, and folic acid as the building blocks for cancer cell division. Nicotinamide adenine dinucleotide (NAD) is a co-enzyme that mediates redox reactions in a number of metabolic pathways, including glycolysis. Increased NAD levels enhance glycolysis and fuel cancer cells. In this context NAD levels depletion subsequently suppress cancer cell proliferation through inhibition of energy production pathways, such as glycolysis, tricarboxylic acid (TCA) cycle, and oxidative phosphorylation. NAD also serves as a substrate for several enzymes thus regulating DNA repair, gene expression, and stress response through these enzymes. Thus, NAD metabolism is implicated in cancer pathogenesis beyond energy metabolism and considered a promising therapeutic target for cancer treatment in particular on cancer cells that displays NAD deficiency due to DNA repair genes deficiency (for example ERCC1 and ATM deficiency) or IDHs (Isocitrate dehydrogenase) mutations.

There also remains a need for new treatment methods to successfully address cancer cell populations without the emergence of cancer cells resistant to therapies.

### SUMMARY OF THE INVENTION

The present invention provides new conjugated nucleic acid molecules which target DNA repair pathways and stimulate the STING pathway specifically in cancer cells. More specifically, the nucleic acid molecule is able to activate PARP without any activation of DNA-PK.

The present invention relates to a conjugated nucleic acid molecule comprising a double-stranded nucleic acid moiety, the 5′end of the first strand and the 3′end of the complementary strand being linked together by a loop, and optionally a molecule facilitating the endocytosis which is linked to the loop,

wherein
the length of the double-stranded nucleic acid moiety is from 10 to 20 base pairs;
the sequence of the double-stranded nucleic acid moiety has less than 80% sequence identity to any gene in a human genome;
the double-stranded nucleic acid moiety comprises deoxyribonucleotides and up to 30% of ribonucleotides or modified deoxyribonucleotides with respect to the total number of nucleotides of the nucleic acid molecule; and
the loop has a structure selected from one of the following formulae:

—O—P(X)OH—O—{[(CH₂)₂—O]_{g}—P(X)OH—O}ᵣ—K—O—P(X)OH—O—{[(CH₂)₂—O]ₕ—P(X)OH—O—}ₛ (I)
with r and s being independently an integer 0 or 1; g and h being independently an integer from 1 to 7 and the sum g+h being from 4 to 7;
with K being

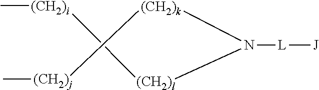

with i, j, k and 1 being independently an integer from 0 to 6, preferably from 1 to 3; or

—O—P(X)OH—O—[(CH₂)_{d}—C(O)—NH]_{b}—CHR—[C(O)—NH—(CH₂)ₑ]_{c}—O—P(X)OH—O— (II)
with b and c being independently an integer from 0 to 4, and the sum b+c is from 3 to 7;
d and e being independently an integer from 1 to 3, preferably from 1 to 2; and
with R being -L_{f}-J,
X being O or S, L being a linker and f being an integer being 0 or 1, and J being a molecule facilitating the endocytosis or being H.

The nucleic acid molecule can comprise one of the following sequences:

(SEQ ID NO 1)
5′ CCCAGCAAACAAGCCT-∫
3′ GGGTCGTTTGTTCGGA-∫
and
(SEQ ID NO 2)
5′ CAGCAACAAG-∫
3′ GTCGTTGTTC-∫

or a sequence wherein 1 to 3 nucleotides are substituted by a ribonucleotide or a modified deoxyribonucleotide or ribonucleotide.

The molecule facilitating the endocytosis can be selected from the group consisting of a cholesterol, single or double chain fatty acids, ligand which targets a cell receptor enabling receptor mediated endocytosis, or a transferrin.

More specifically, the molecule facilitating the endocytosis is a cholesterol.

Alternatively, the molecule facilitating the endocytosis is a ligand of a sigma-2 receptor (σ2R). For instance, the ligand of a sigma-2 receptor (σ2R) comprises the following formula:

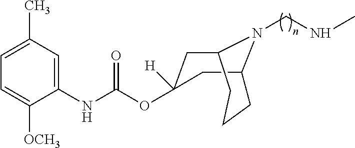

with n being an integer from 1 to 20.

In an aspect, 1, 2 or 3 internucleotidic linkages of the nucleotides located at the free end of the double-stranded moiety of the nucleic acid molecule can have a modified phosphodiester backbone such as a phosphorothioate linkage, preferably on both strands. For instance, 1 to 3 thymines can be replaced by 2′-deoxy-2′-fluoroarabinouridine, or 1 to 3 guanosines can be replaced by 2′-deoxy-2′-fluoroarabinoguanosine; or 1 to 3 cytidines can be replaced by 2′-deoxy-2′-fluoroarabinocytidine.

The loop can have the formula (I) and K is

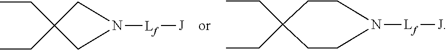

Optionally, f is 1 and L-J is selected in the group consisting of —C(O)—(CH₂)ₘ—NH—[(CH₂)₂—O]ₙ—(CH₂)ₚ—C(O)-J, —C(O)—(CH₂)ₘ—NH—C(O)—[(CH₂)₂—O]ₙ—(CH₂)ₚ-J, C(O)—(CH₂)ₘ NH—C(O)—CH₂—O—[(CH₂)₂—O]ₙ—(CH₂)ₚ-J, —C(O)—(CH₂)ₘ—NH—C(O)—[(CH₂)₂—O]ₙ—(CH₂)ₚ—C(O)-J and —C(O)—(CH₂)ₘ—NH—C(O)—CH₂—O—[(CH₂)₂—O]ₙ—(CH₂)ₚ—C(O)-J, with m being an integer from 0 to 10; n being an integer from 0 to 15; and p being an integer from 0 to 3.

Optionally, the loop has the formula (I)

—O—P(X)OH—O—{[(CH₂)₂-]_{g}—P(X)OH—O}ᵣ—K—O—P(X)OH—O—{[(CH₂)₂—O]ₕ—P(X)OH—O—}ₛ (I)

with X being S, r being 1, g being 6, s being 0, and K being

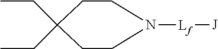

with f being 1 and L being C(O)—(CH₂)₅—NH—[(CH₂)₂—O]₃—(CH₂)₂—C(O)-J, —C(O)—(CH₂)₅—NH—C(O)—[(CH₂)₂—O]₃—(CH₂)₃-J, —C(O)—(CH₂)₅—NH—C(O)—CH₂—O—[(CH₂)₂—O]₅—CH₂—C(O)-J, —C(O)—(CH₂)₅—NH—C(O)—CH₂—O—[(CH₂)₂—O]₉—CH₂—C(O)-J, —C(O)—(CH₂)₅—NH—C(O)—CH₂—O—[(CH₂)₂—O]₁₃—CH₂—C(O)-J, or —C(O)—(CH₂)₅—NH—C(O)-J.

Optionally, f is 1 and L-J is —C(O)—(CH₂)ₘ—NH—[C(O)]t-[(CH₂)₂—O]ₙ—(CH₂)ₚ—[C(O)]ᵥ-J or —C(O)—(CH₂)ₘ—NH—[C(O)—CH₂—O]ₜ—[(CH₂)₂—O]ₙ—(CH₂)ₚ—[C(O)]ᵥ-J with m being an integer from 0 to 10; n being an integer from 0 to 15; p being an integer from 0 to 4; t and v being an integer 0 or 1 with at least one among t and v being 1.

In one particular aspect, L can be selected in the group consisting of —C(O)—(CH₂)ₘ NH—[(CH₂)₂₋₀]ₙ—(CH₂)ₚ—C(O)-J, —C(O)—(CH₂)ₘ—NH—C(O)—[(CH₂)₂—O]ₙ—(CH₂)ₚ-J, C(O)—(CH₂)ₘ—NH—C(O)—CH₂—O—[(CH₂)₂—O]ₙ—(CH₂)ₚ-J, —C(O)—(CH₂)ₘ—NH—C(O)—[(CH₂)₂—O]ₙ—(CH₂)ₚ—C(O)-J and —C(O)—(CH₂)ₘ—NH—C(O)—CH₂—O—[(CH₂)₂—O]ₙ—(CH₂)ₚ—C(O)-J, with m being an integer from 0 to 10; n being an integer from 0 to 15; and p being an integer from 0 to 3.

In a very specific aspect, L can be selected in the group consisting of —C(O)—(CH₂)₅—NH—[(CH₂)₂—O]₃—(CH₂)₂—C(O)-J, —C(O)—(CH₂)₅—NH—C(O)—[(CH₂)₂—O]₃—(CH₂)₃-J, —C(O)—(CH₂)₅—NH—C(O)—CH₂—O—[(CH₂)₂—O]₅—CH₂—C(O)-J, —C(O)—(CH₂)₅—NH—C(O)—CH₂—O—[(CH₂)₂—O]₉—CH₂—C(O)-J, —C(O)—(CH₂)₅—NH—C(O)—CH₂—O—[(CH₂)₂—O]₁₃—CH₂—C(O)-J, or —C(O)—(CH₂)₅—NH—C(O)-J.

In a particular aspect, the conjugated nucleic acid molecule is selected from the group consisting of

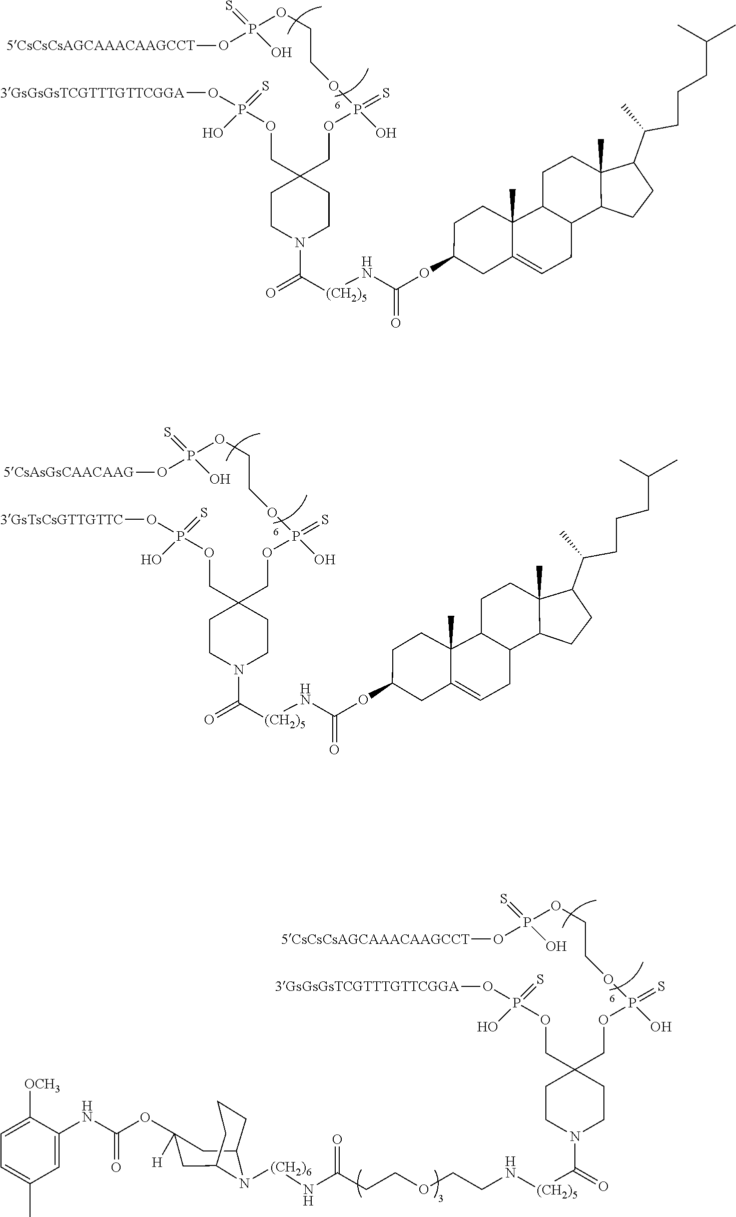

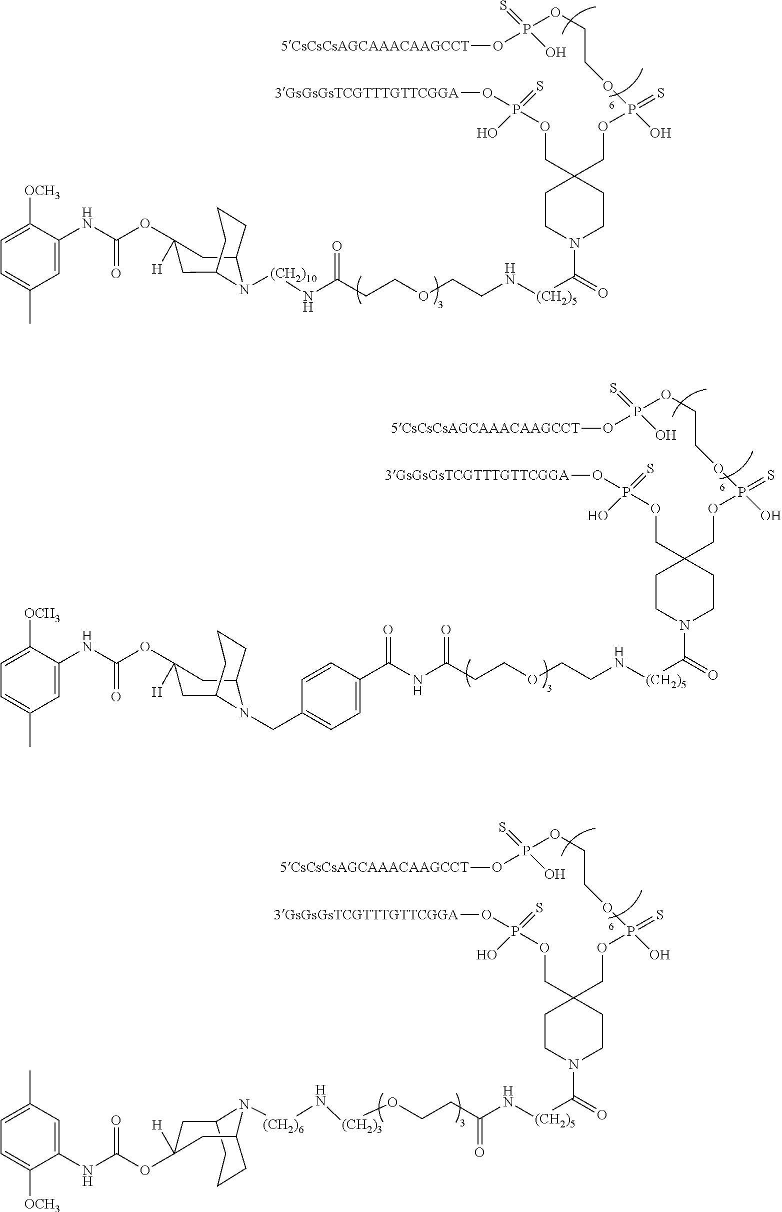

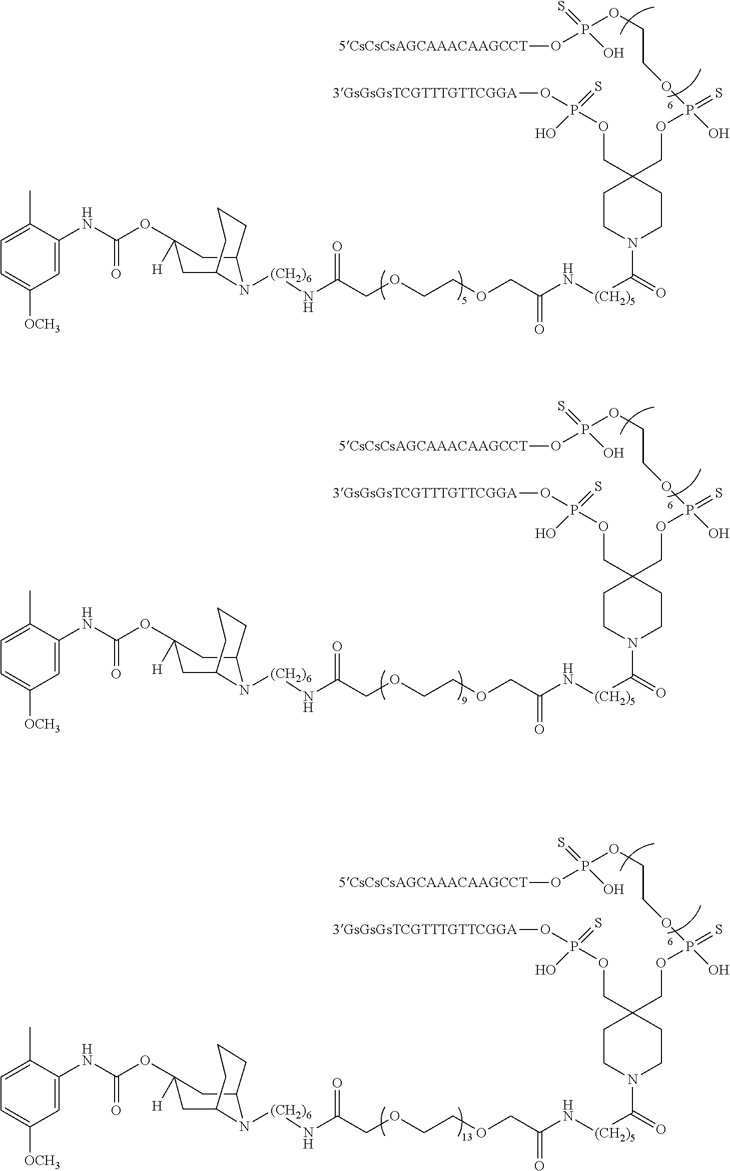

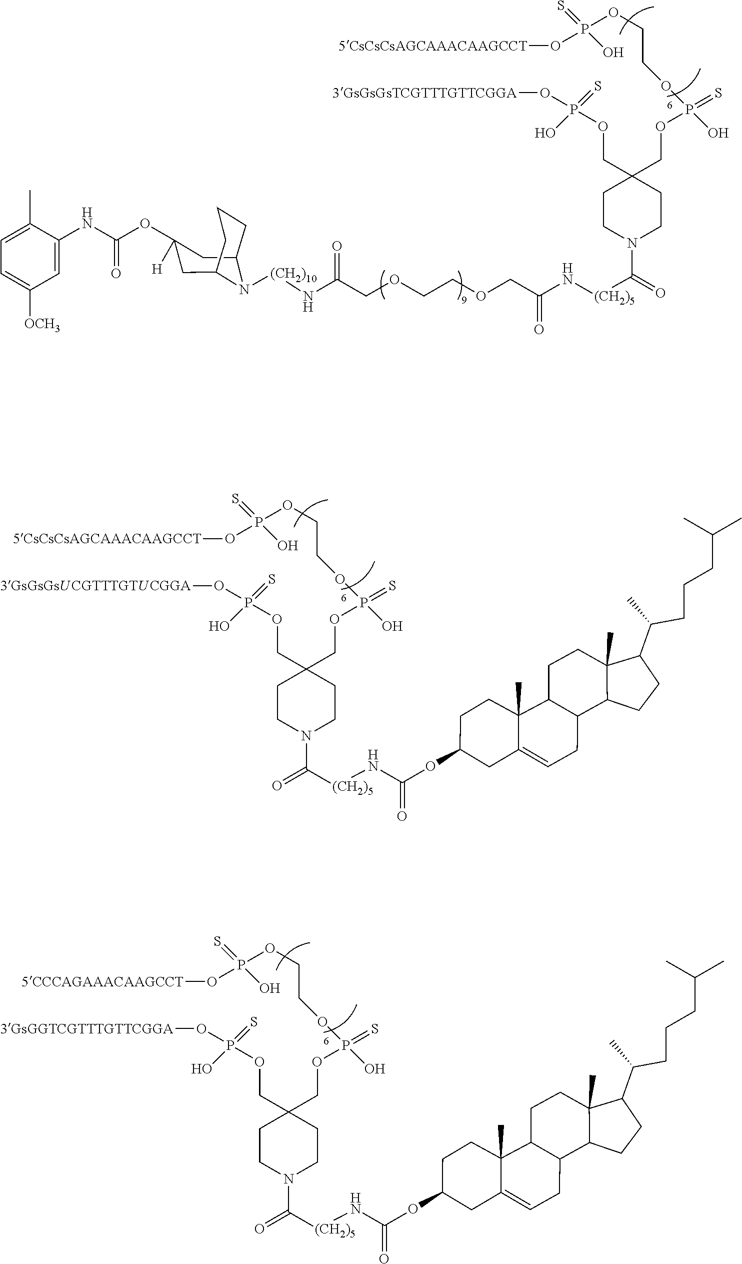

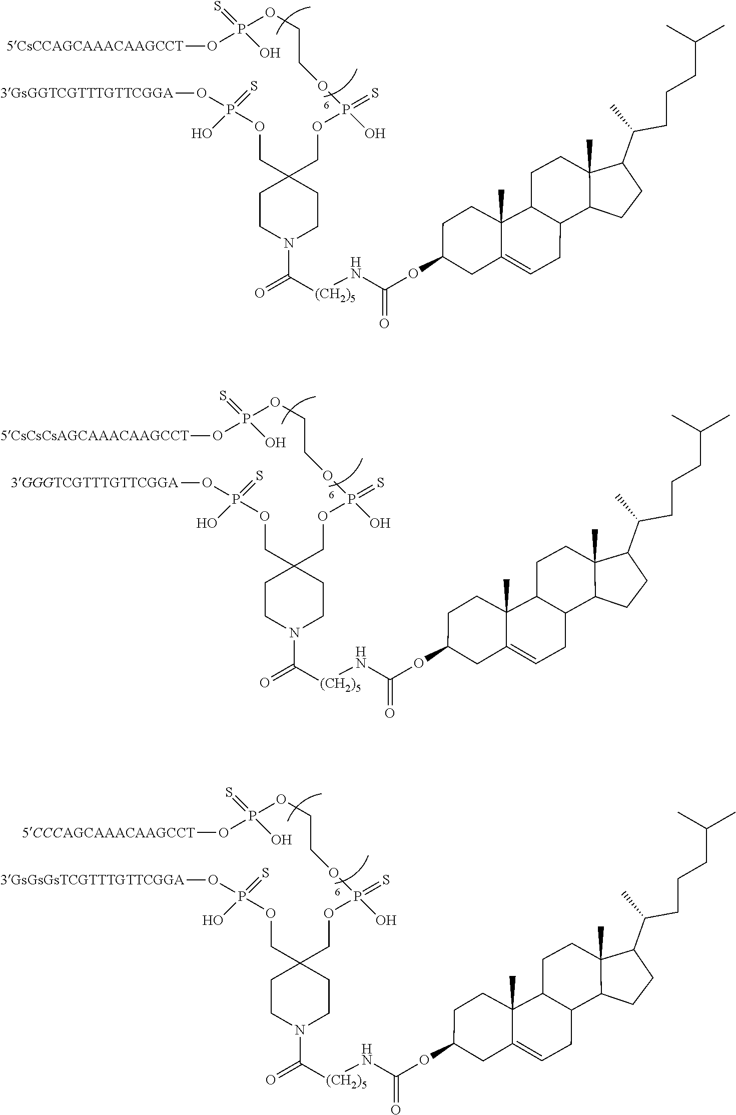

wherein internucleotide linkages “s” refers to phosphorothioate internucleotide linkages; italic U being 2′-deoxy-2′-fluoroarabinouridine, italic G being 2′-deoxy-2′-fluoroarabinoguanosine; italic C being 2′-deoxy-2′-fluoroarabinocytidine; or the pharmaceutically acceptable salts thereof.

The present invention also relates to a pharmaceutical composition comprising a conjugated nucleic acid molecule according to the present disclosure. Optionally, the pharmaceutical composition further comprises an additional therapeutic agent, preferably selected from an immunomodulator such as an immune checkpoint inhibitor (ICI), a T-cell-based cancer immunotherapy such as adoptive cell transfer (ACT), genetically modified T-cells or engineered T-cells such as chimeric antigen receptor cells (CAR-T cells), or a conventional chemotherapeutic, radiotherapeutic or anti-angiogenic agent, HDAC inhibitor (such as belinostat) or targeted immunotoxin.

The present invention also relates to a conjugated nucleic acid molecule or a pharmaceutical composition according to the present disclosure for use as a drug, in particular for use for the treatment of cancer. It further relates to a method of treating a cancer in a subject in need thereof, comprising administering a therapeutically efficient amount of a conjugated nucleic acid molecule or a pharmaceutical composition according to the present invention, repeatedly or chronically. Optionally, the method comprises administering repeated cycles of treatment, preferably for at least two cycles of administration, even more preferably at least three or four cycles of administration.

Repeated or chronic administrations of a conjugated nucleic acid molecule according to the invention does not lead cancer cells to develop resistance to the therapy. It can be used in combination with an immunomodulator, such as an immune checkpoint inhibitor (ICI), or in combination with T-cell-based cancer immunotherapy including adoptive cell transfer (ACT), genetically modified T-cells or engineered T-cells such as chimeric antigen receptor cells (CAR-T cells).

Accordingly, the conjugated nucleic acid molecule or the pharmaceutical composition is for use in the treatment of cancer, in combination with an additional therapeutic agent, preferably selected from an immunomodulator such as an immune checkpoint inhibitor (ICI), a T-cell-based cancer immunotherapy such as adoptive cell transfer (ACT), genetically modified T-cells or engineered T-cells such as chimeric antigen receptor cells (CAR-T cells), or a conventional chemotherapeutic, radiotherapeutic or anti-angiogenic agent, HDAC inhibitor (such as belinostat) or targeted immunotoxin.

In a particular aspect, the present invention also relates to a way for a possible selection strategy or a clinical stratification strategy for patients with tumors carrying deficiencies in the NAD⁺ synthesis. These patients could be better responders for the drug treatment according to the present invention, in particular patients with tumors carrying both DNA repair pathways deficiencies (for example ERCC1 and ATM deficiency) or IDHs mutations.

In a particular aspect, the conjugated nucleic acid molecule or the pharmaceutical composition is for use for a targeted effect against tumor cells carrying deficiencies in the NAD⁺ synthesis in the treatment of cancer. More particularly, the tumor cells further carry DNA repair pathways deficiencies selected from ERCC1 or ATM deficiency or IDHs mutations.

### BRIEF DESCRIPTION OF THE DRAWINGS

FIG. 1: OX401-induced target engagement. Cells were treated for 24 hours with increasing doses of OX401 or AsiDNA™ and assessed for (A) DNA-PK activation through H2AX phosphorylation (γH2AX) and (B) PARP hyperactivation by measuring cellular PARylation (by detecting Poly(ADP-Ribose) (PAR) polymers). ***, p<0.001.

FIG. 2: OX401 displays tumor specific cytotoxicity. (A) Tumor cells and (B) non-tumor cells were treated with OX401 or AsiDNA™ and cell survival was assessed using an XTT assay. Cell survival was calculated as the ratio of living treated cells to living not-treated cells. IC50 were calculated according to the dose-response curves using GraphPadPrism software.

FIG. 3: OX401 triggers a tumor immune response. MDA-MB-231 cells treated for a long term with OX401 or AsiDNA™ were assessed for (A) the % of micronuclei positive cells, (B) the amount of secreted CCL5 and CXCL10 chemokines using ELISA assays and the level of (C) total PD-L1 by western blot and (D) surface-associated PD-L1 by flow cytometry analysis. cGAMP, STING agonist; **, p<0.01.

FIG. 4: OX402 induces PARP activation. Cells were treated for 24 hours with increasing doses of OX402 and assessed for PARP hyperactivation by measuring cellular PARylation (by detecting Poly(ADP-Ribose) (PAR) polymers).

FIG. 5: OX401 induces PARylation and efficient NAD⁺ depletion in tumor cells. Cells were treated during 48 hours, 7 days or 13 days with OX401 (5 μM) and assessed for PARP hyperactivation by western blot analysis of PARylated proteins (A, D), NAD⁺ intracellular levels (B, E) and cell survival (C, F). % of NAD⁺ and survival are expressed as a ratio of treated cells to non-treated cells (NT). (A, B, C) MDA-MB-231 tumor cells, (D, E, F) MRC5 lung fibroblasts.

FIG. 6: OX401 abrogates the homologous recombination repair pathway. Cells were treated for 48 hours with OX401 (5 μM) and levels of DSBs assessed using (A) flow cytometry to detect the phosphorylated form of H2AX (γH2AX) or (B) immunofluorescence to detect γH2AX Foci. (C-D) Efficacy of the homologous recombination pathway after olaparib (5 μM) treatment with or without OX401 (5 μM) for 48 hours was analyzed by (C) the detection of Rad51 protein recruitment to sites of DSBs and (D) quantification of Rad51 Foci. ***, p<0.001.

FIG. 7: Tumor cells treated with OX401 do not develop resistance. (A) Cells were treated with Talazoparib (2 μM) or OX401 (1.5 μM) and counted after every treatment and amplification cycle. (B) Cell survival was estimated by dividing the number of treated cells by the mean number of untreated cells and determined after each period of treatment. (C) Resistance to Talazoparib was validated in the three isolated populations (Ta11, Ta12 and Ta13) compared to U937 parental cells using an XTT assay 4 days after treatment with increased doses of Talazoparib. The survival percentage was normalized with the non-treated condition.

FIG. 8: OX401 potentiates the anti-tumor immune response. MDA-MB-231 cells co-cultured with T lymphocytes (ratio effector cells to target tumor cells 4:1) with or without OX401 (5 μM) for 48 hours were assessed for (A) tumor cells proliferation, (B) the amount of secreted Granzyme B enzyme using ELISA assay and (C, D) the activation of the STING pathway by western blot (C) or ELISA assay to quantify the secreted CCL5 chemokine (D). LTₐ, activated T lymphocytes; MDA, MDA-MB-231 tumor cells.

FIG. 9: Kinetics of association (kₒₙ) and strength of interaction (K_{D}) of OX401, OX402, OX406, OX407, OX408, OX410 and OX411, with PARP-1.

### DETAILED DESCRIPTION OF THE INVENTION

The present invention relates to new nucleic acid molecules conjugated to a molecule facilitating the endocytosis such as cholesterol-nucleic acid conjugates, which target and activate specifically PARPs, inducing a profound down regulation of cellular NAD and therefore particularly dedicated for cancer treatment, in particular on cancer cells that display NAD deficiency due to DNA repair genes deficiency (for example ERCC1 and ATM deficiency) or IDHs (Isocitrate dehydrogenase) mutations.

The present invention relates to new nucleic acid molecules conjugated to a molecule facilitating the endocytosis such as cholesterol-nucleic acid conjugates, which target DDR mechanisms and are also STING agonists allowing their combination with immune checkpoint therapy (ICT) for an optimal treatment of cancer.

Accordingly, the inventors surprisingly found that:
1) The activation of PARP without activation of DNA-PK by the conjugated nucleic acid molecules of the present invention leads to an increase of cancer cells with micronuclei, cytoplasmic chromatin fragments (CCF) and cytotoxicity by standalone use in comparison with Dbait molecules.
2) The specific increase of micronuclei (MN) and cytoplasmic chromatin fragments (CCF) in cancer cells leads to an early increase of STING pathway activation as shown by the increase of inflammatory cytokines (CXCL10 and CCL5) release and PD-L1 and NKG2s expression on cancer cells. These effects are specific to cancer cells. Such a cancer cell specificity precludes general and extensive inflammation with subsequent deleterious possible side effects.
3) The activation of the STING pathway through DNA repair pathway inhibition and generation of either micronuclei and CCFs represent a very attractive way to specifically activate the STING pathway in tumor cells, in particular by innate immunity activation.

Based on these observations, the present invention relates to:
a conjugated nucleic acid molecule as described below;
a pharmaceutical composition comprising a conjugated nucleic acid molecule as described below and a pharmaceutically acceptable carrier, in particular for use in the treatment of cancer;
a conjugated nucleic acid molecule as described below for use as a drug, in particular for use in the treatment of cancer;
the use of a conjugated nucleic acid molecule as described below for the manufacture of a drug, in particular for use in the treatment of cancer;
a method for treating a cancer in a patient in need thereof, comprising administering an effective amount of a conjugated nucleic acid molecule as disclosed herein;
a pharmaceutical composition comprising a conjugated nucleic acid molecule as described below, an additional therapeutic agent and a pharmaceutically acceptable carrier, in particular for use in the treatment of cancer;
a product or kit containing (a) a conjugated nucleic acid molecule as disclosed below, and optionally b) an additional therapeutic agent, as a combined preparation for simultaneous, separate or sequential use, in particular in the treatment of cancer;
a combined preparation which comprises (a) a hairpin nucleic acid molecule as disclosed below, b) an additional therapeutic agent as described below for simultaneous, separate or sequential use, in particular in the treatment of cancer;
a pharmaceutical composition comprising a conjugated nucleic acid molecule as disclosed below, for the use in the treatment of cancer in combination with an additional therapeutic agent;
the use of a pharmaceutical composition comprising a conjugated nucleic acid molecule as disclosed below for the manufacture of a medicament for the treatment of cancer in combination with an additional therapeutic agent;
a method for treating a cancer in a patient in need thereof, comprising administering an effective amount of a) a conjugated nucleic acid molecule as disclosed below, and b) an effective amount of an additional therapeutic agent;
a method for treating a cancer in a patient in need thereof, comprising administering an effective amount of a pharmaceutical composition comprising a conjugated nucleic acid molecule as disclosed herein, and an effective amount of an additional therapeutic agent;
a method for increasing the efficiency of a treatment of a cancer with a therapeutic antitumor agent, or for enhancing tumor sensitivity to treatment with a therapeutic antitumor agent in a patient in need thereof, comprising administering an effective amount of a conjugated nucleic acid molecule as disclosed below;
a method for treating cancer comprising administering a conjugated nucleic acid molecule as disclosed herein, repeatedly or chronically, by repeated cycles of treatment, preferably for at least two cycles of administration, even more preferably at least three or four cycles of administration;
a method of treating cancer in patients with tumor cells carrying deficiencies in the NAD+ synthesis, and optionally DNA repair pathways deficiencies selected from ERCC1 or ATM deficiency or IDHs mutations.

### Definitions

Whenever within this whole specification “treatment of a cancer” or the like is mentioned with reference to the pharmaceutical composition, kit, product and combined preparation of the invention, there is meant: a) a method for treating a cancer, said method comprising administering a pharmaceutical composition, kit, product and combined preparation of the invention to a patient in need of such treatment; b) a pharmaceutical composition, kit, product and combined preparation of the invention for use in the treatment of a cancer; c) the use of a pharmaceutical composition, kit, product and combined preparation of the invention for the manufacture of a medicament for the treatment of a cancer; and/or d) a pharmaceutical composition, kit, product and combined preparation of the invention for use in the treatment a cancer.

Within the context of the invention, the term “treatment” denotes curative, symptomatic, and preventive treatment. Pharmaceutical compositions, kits, products and combined preparations of the invention can be used in humans with existing cancer or tumor, including at early or late stages of progression of the cancer. The pharmaceutical compositions, kits, products and combined preparations of the invention will not necessarily cure the patient who has the cancer but will delay or slow the progression or prevent further progression of the disease, improving thereby the patients' condition. In particular, the pharmaceutical compositions, kits, products and combined preparations of the invention reduce the development of tumors, reduce tumor burden, produce tumor regression in a mammalian host and/or prevent metastasis occurrence and cancer relapse. In treating the cancer, the pharmaceutical composition, kit, product and combined preparation of the invention is administered in a therapeutically effective amount.

The terms “kit”, “product” or “combined preparation”, as used herein, define especially a “kit-of-parts” in the sense that the combination partners (a) and (b), as defined above can be dosed independently or by use of different fixed combinations with distinct amounts of the combination partners (a) and (b), i.e. simultaneously or at different time points. The components of the kit-of-parts can then, e.g., be administered simultaneously or chronologically staggered, that is at different time points and with equal or different time intervals for any part of the kit-of-parts. The ratio of the total amounts of the combination partner (a) to the combination partner (b), to be administered in the combined preparation can be varied. The combination partners (a) and (b) can be administered by the same route or by different routes.

By “effective amount” it is meant the quantity of the pharmaceutical composition, kit, product and combined preparation of the invention which prevents, removes or reduces the deleterious effects of cancer in mammals, including humans, alone or in combination with the other active ingredients of the pharmaceutical composition, kit, product or combined preparation. It is understood that the administered dose may be adapted by those skilled in the art according to the patient, the pathology, the mode of administration, etc.

The term “STING” refers to STtimulator of INterferon Genes receptor, also known as TMEM173, ERIS, MITA, MPYS, SAVI, or NET23). As used herein, the terms “STING” and “STING receptor” are used interchangeably, and include different isoforms and variants of STING. The mRNA and protein sequences for human STING isoform 1, the longest isoform, have the NCBI Reference Sequence [NM_198282.3] and [NP_938023.1]. The mRNA and protein sequences for human STING isoform 2, a shorter isoform have the NCBI Reference Sequence [NM_001301738.1] and [NP_001288667.1].

The term “STING activator”, as used herein, refers to a molecule capable of activating the STING pathway. Activation of the STING pathway may include, for example, stimulation of inflammatory cytokines, including interferons, such as type 1 interferons, including IFN-α, IFN-β, type 3 interferons, e.g., IFN-λ, IP-10 (interferon-7-inducible protein also known as CXCL10), PD-L1, TNF, IL-6, CXCL9, CCL4, CXCL11, NKG2D ligand (MICA/B), CCL5, CCL3, or CCL8. Activation of the STING pathway may also include stimulation of TANK binding kinase (TBK) 1 phosphorylation, interferon regulatory factor (IRF) activation (e.g., IRF3 activation), secretion of IP-10, or other inflammatory proteins and cytokines. Activation of the STING pathway may be determined, for example, by the ability of a compound to stimulate activation of the STING pathway as detected using an interferon stimulation assay, a reporter gene assay (e.g., a hSTING wt assay, or a THP-1 Dual assay), a TBK1 activation assay, IP-10 assay, or other assays known to persons skilled in the art. Activation of the STING pathway may also be determined by the ability of a compound to increase the level of transcription of genes that encode proteins activated by STING or the STING pathway. Such activation may be detected, for example, using an RNAseq assay.

Activation of the STING pathway can be determined by one or more “STING assays” selected from: an interferon stimulation assay, a hSTING wt assay, a THP1-Dual assay, a TANK binding kinase 1 (TBK1) assay, an interferon-γ-inducible protein 10 (IP-10) secretion assay or a PD-L1 assay.

More specifically, a molecule is a STING activator if it is able to stimulate production of one or more STING-dependent cytokines in a STING-expressing cell at least 1.1-fold, 1.2-fold, 1.3-fold, 1.4-fold, 1.5-fold, 1.6-fold, 1.7-fold, 1.8-fold, 1.9-fold, 2-fold or greater than an untreated STING-expressing cell. Preferably, the STING-dependent cytokine is selected from interferon, type 1 interferon, IFN-α, IFN-β, type 3 interferon, IFN-λ, CXCL10 (IP-10), PD-L1 TNF, IL-6, CXCL9, CCL4, CXCL11, NKG2D ligand (MICA/B), CCL5, CCL3, or CCL8, more preferably CCL5 or CXCL10.

Conjugated Nucleic Acid Molecules

An additional advantage of some of the conjugated nucleic acid molecules according to the present invention is based on the fact that they can be synthesized as one molecule by only using oligonucleotide solid phase synthesis, thereby allowing low costs and a high manufacturing scale.

The conjugated nucleic acid molecule of the present invention comprises a double-stranded nucleic acid moiety, the 5′end of the first strand and the 3′end of the complementary strand being linked together by a loop, and optionally a molecule facilitating the endocytosis which is linked to the loop. The other end of the double-stranded nucleic acid moiety is free.

Conjugated nucleic acid molecules according to the present invention may be defined by a number of characteristics necessary for their therapeutic activity, such as their minimal and maximal length, the presence of at least one free end, and the presence of a double stranded portion, preferably a double-stranded DNA portion.

The conjugated nucleic acid molecule is capable of activating PARP-1 protein. On the other hand, the conjugated nucleic acid molecule does not activate DNA-PK.

The present invention also relates to a pharmaceutically acceptable salt of the conjugated nucleic acid molecule of the present invention

Nucleic Acid Molecules

The length of the conjugated nucleic acid molecules may be variable, as long as it is sufficient to allow appropriate binding and activation of PARP (PARP-1) protein and it is insufficient to allow appropriate binding of Ku protein complex comprising Ku and DNA-PKcs proteins. As it has been shown that the length of conjugated nucleic acid molecules must be greater than 20 bp, preferably about 32 bp, to ensure binding to such a Ku complex and allowing DNA-PKcs activation, the length is up to 20 bp. In addition, it has been shown that the length of conjugated nucleic acid molecules must be greater than 8 bp for allowing appropriate binding and activation of PARP.

The length of the double-stranded nucleic acid moiety is from 10 to 20 base pairs. A length of at most 20 bp prevents the molecule from being able to activate DNA-PK. In a particular aspect, the length of the double-stranded nucleic acid moiety is from 11 to 19 base pairs. For instance, the length could be from 11 to 19 bp, 12 to 19 bp, 13 to 19 bp, 14 to 19 bp, 15 to 19 bp, 16 to 19 bp, 12 to 16 bp, 12 to 17 bp, 12 to 18 bp, 13 to 16 bp, 13 to 17 bp, 13 to 18 bp, 14 to 16 bp, 14 to 17 bp, 14 to 18 bp, 15 to 16 bp, 15 to 17 bp or 15 to 18 bp. In a very particular aspect, the length of the double-stranded nucleic acid moiety is 16 bp. By “bp” is intended that the molecule comprise a double stranded portion of the indicated length.

The effect of the nucleic acid molecules does not depend on its sequence.

Accordingly, the nucleic acid molecule could be defined as comprising the following formula

5′ NNNN(N)ₐN-∫

3′ NNNN(N)ₐN-∫

wherein N is a nucleotide, “a” is an integer from 5 to 15, and the two strands are complementary to each other. “−∫” indicates that the nucleotide is linked to the loop. In a particular aspect, “a” is an integer from 6 to 14. In another particular aspect, “a” can be an integer from 6 to 14, 7 to 14, 8 to 14, 9 to 14, 10 to 14, 11 to 14, 6 to 13, 7 to 13, 8 to 13, 9 to 13, 10 to 13, 11 to 13, 6 to 12, 7 to 12, 8 to 12, 9 to 12, or 10 to 12.

Preferably, the sequence of the nucleic acid molecule is of non-human origin (i.e., their nucleotide sequence and/or conformation does not exist as such in a human cell). The conjugated nucleic acid molecules have preferably no significant degree of sequence homology or identity to known genes, promoters, enhancers, 5′- or 3′-upstream sequences, exons, introns, and the like. In other words, the conjugated nucleic acid molecules have less than 80% or 70%, even less than 60% or 50% sequence identity to any gene in a human genome. Methods of determining sequence identity are well known in the art and include, e.g., BLASTN 2.2.25. For instance, the identity percentage can be determined with the Human Genome Build 37 (reference GRCh37.p2 and alternate assemblies). The conjugated nucleic acid molecules do not hybridize, under stringent conditions, with human genomic DNA. Typical stringent conditions are such that they allow the discrimination of fully complementary nucleic acids from partially complementary nucleic acids.

In addition, the sequence of the conjugated nucleic acid molecules is preferably devoid of 5′-CpG-3′ in order to avoid the well-known toll-like receptor (TLR)-mediated immunological reactions.

The conjugated nucleic acid molecules must have one free end, as a mimic of double-stranded break. Said free end may be either a free blunt end or a 5′-/3′-protruding end. The “free end” refers herein to a nucleic acid molecule, in particular a double-stranded nucleic acid moiety having both a 5′ end and a 3′ end.

For instance, the double-stranded nucleic acid moiety or the nucleic acid of the molecule according to the present invention comprises or consists in the following sequence (SEQ ID NO: 1):

5′ CCCAGCAAACAAGCCT-∫
3′ GGGTCGTTTGTTCGGA-∫

In a particular embodiment, the conjugated nucleic acid molecule has a double stranded moiety comprising the same nucleotide sequence as SEQ ID NO: 1. Optionally, the conjugated nucleic acid molecule has the same nucleotide composition as SEQ ID NO: 1 but the nucleotide sequence is different. Then, the conjugated nucleic acid molecule comprises one strand of the double stranded moiety with 6 A, 7 C, 2 G and 1 T. Preferably, the sequence of the conjugated nucleic acid molecules does not contain any 5′-CpG-3′ dinucleotide. Alternatively, the double stranded moiety comprises at least 9, 10, 11, 12, 13, 14, 15, or 16 consecutive nucleotides of SEQ ID NO: 1. In a more particular embodiment, the double stranded moiety consists of 9, 10, 11, 12, 13, 14, 15, or 16 consecutive nucleotides of SEQ ID NO: 1.

In another particular aspect the double-stranded nucleic acid moiety or the nucleic acid of the molecule according to the present invention comprises or consists in the following sequence (SEQ ID NO: 2):

5′ CAGCAACAAG-∫
3′ GTCGTTGTTC-∫

In a particular embodiment, the conjugated nucleic acid molecule has a double stranded moiety comprising the same nucleotide sequence as SEQ ID NO: 2. Optionally, the conjugated nucleic acid molecule has the same nucleotide composition as SEQ ID NO: 2 but the nucleotide sequence is different. Then, the conjugated nucleic acid molecule comprises one strand of the double stranded moiety with 5 A, 3 C and 2 G. Preferably, the sequence of the conjugated nucleic acid molecules does not contain any 5′-CpG-3′ dinucleotide.

The double-stranded nucleic acid moiety may comprise nucleotide(s) with a modified phosphodiester backbone, in particular in order to protect them from degradation. Preferably, the nucleotide(s) having a modified phosphodiester backbone are located at the free end of the 20 double-stranded moiety of the nucleic acid molecule. In one aspect, 1, 2 or 3 internucleotidic linkages of the nucleotides located at the free end of the double-stranded moiety of the nucleic acid molecule have a modified phosphodiester backbone, preferably on both strands. Alternatively, preferred the conjugated nucleic acid molecules have a 3′-3′ nucleotide linkage at the end of a strand.

In a particular embodiment, the nucleic acid molecule could be defined as comprising the following formula

NNNN(N)ₐN-∫
NNNN(N)ₐN-∫

wherein the internucleotidic linkages of underlined nucleotides N have a modified phosphodiester backbone.

For instance, the double-stranded nucleic acid moiety or the nucleic acid of the molecule according to the present invention comprises or consists in the following sequence (SEQ ID NO: 1):

5′ CCCAGCAAACAAGCCT-∫
3′ GGGTCGTTTGTTOGGA-∫

wherein the internucleotidic linkages of underlined nucleotides N have a modified phosphodiester backbone.

In another particular aspect, the double-stranded nucleic acid moiety or the nucleic acid of the molecule according to the present invention comprises or consists in the following sequence (SEQ ID NO: 2):

5′ CAGCAACAAG-∫
3′ GTCGTTGTTC-∫

wherein the internucleotidic linkages of underlined nucleotides N have a modified phosphodiester backbone.

The modified phosphodiester backbone can be a phosphorothioate backbone.

When the modified phosphodiester linkage is a phosphorothioate linkage, the molecule could be the followings:

5′CsCsCsAGCAAACAAGCCT-∫
3′GsGsGsTCGTTTGTTCGGA-∫
or
5′CsAsGsCAACAAG-∫
3′GsTsCsGTTGTTC-∫

In an alternative aspect, the double-stranded nucleic acid moiety may comprise one modified phosphodiester linkage, e.g., a phosphorothioate linkage, on the two last nucleotides at the 3′ end of the molecule; on the two last nucleotides at the 5′ end of the molecule; or on the two last nucleotides both at the 3′ end and at the 5′ end of the molecule.

For instance, the double-stranded nucleic acid moiety comprises or consists in a moiety selected from the followings:

5′CCCAGCAAACAAGCCT-∫
3′GGGTCGTTTGTTCGGA-∫
5′CAGCAACAAG-∫
3′GTCGTTGTTC-∫
5′CCCAGCAAACAAGCCT-∫
3′GGGTCGTTTGTTCGGA-∫
and
5′CAGCAACAAG-∫
3′GTCGTTGTTC-∫.

When the modified phosphodiester linkage is a phosphorothioate linkage, the molecule could be the followings:

5′CCCAGCAAACAAGCCT-∫
3′GsGGTCGTTTGTTCGGA-∫
5′CAGCAACAAG-∫
3′GsTCGTTGTTC-∫
5′CsCCAGCAAACAAGCCT-∫
3′GsGGTCGTTTGTTCGGA-∫
and
5′CsAGCAACAAG-∫
3′GsTCGTTGTTC-∫.

In another alternative aspect, the double-stranded nucleic acid moiety may comprise three modified phosphodiester linkage, e.g., a phosphorothioate linkage, on the three last nucleotides at the 3′ end of the molecule; or on the four last nucleotides at the 5′ end of the molecule.

For instance, the double-stranded nucleic acid moiety comprises or consists in a moiety selected from the followings:

5′CCCAGCAAACAAGCCT-∫
3′GGGTCGTTTGTTCGGA-∫
5′CAGCAACAAG-∫
3′GTCGTTGTTC-∫
5′CCCAGCAAACAAGCCT-∫
3′GGGTCGTTTGTTCGGA-∫
and
5′CAGCAACAAG-∫
3′GTCGTTGTTC-∫.

When the modified phosphodiester linkage is a phosphorothioate linkage, the molecule could be the followings:

5′CsCsCsAGCAAACAAGCCT-∫
3′GGGTCGTTTGTTCGGA-∫
5′CsAsGsCAACAAG-∫
3′GTCGTTGTTC-∫
5′CCCAGCAAACAAGCCT-∫
3′GsGsGsTCGTTTGTTCGGA-∫
and
5′CAGCAACAAG-∫
3′GsTsCsGTTGTTC-∫.

The double-stranded nucleic acid moiety essentially comprises deoxyribonucleotides. However, it may also include some ribonucleotides or modified deoxyribonucleotides or ribonucleotides. In one aspect, the double-stranded nucleic acid moiety only comprises deoxyribonucleotides. In another aspect, the double-stranded nucleic acid moiety comprises deoxyribonucleotides and up to 30, 20, 15 or 10% of ribonucleotides or modified deoxyribonucleotides with respect to the total number of nucleotides of the nucleic acid molecule. In a particular aspect, the double-stranded nucleic acid moiety comprises a first strand comprising only deoxyribonucleotides and a complementary strand carrying the ribonucleotides or modified deoxyribonucleotides. According to one embodiment, the conjugated nucleic acid molecules comprise a modification corresponding to position 2 of the ribose. For instance, the conjugated nucleic acid molecules may comprise at least one 2′-modified nucleotide, e.g., having a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAE), 2′-O-dimethylaminopropyl (2′-O-DMAP), 2′-O-dimethylaminoethyloxyethyl (2′-O-DMAEOE) or 2′-O—N-methylacetamido (2′-O-NMA) modification or e.g. a 2′-deoxy-2′-fluoroarabinonucleotide (FANA). However, such 2′-modified nucleotides are preferably not located at the 5′ or 3′ end of a strand.

In a particular aspect, the conjugated nucleic acid molecules have at least one, two, three or more 2′-deoxy-2′-fluoroarabinonucleotides (FANA). FANA adopts a DNA-like structure resulting in an unaltered recognition of the conjugated nucleic acid molecules by the proteins of interest. FANA include the following pyrimidine 2′-fluoroarabinonucleosides and purine 2′-fluoroarabinonucleosides:
9-(2-Deoxy-2-fluoro-ß-D-arabinofuranosyl)adenine (2′-FANA-A);
9-(2-Deoxy-2-fluoro-ß-D-arabinofuranosyl)guanine (2′-FANA-G);
1-(2-Deoxy-2-fluoro-ß-D-arabinofuranosyl)cytosine (2′-FANA-C);
1-(2-Deoxy-2-fluoro-ß-D-arabinofuranosyl)uracil (2′-FANA-U).

For instance, the double-stranded nucleic acid moiety or the nucleic acid of the molecule according to the present invention comprises or consists in the following sequence (SEQ ID NO: 1):

5′CCCAGCAAACAAGCCT-∫
3′GGG [symbol] CGTTTGT [symbol] CGGA-∫

wherein the U is 1-(2-deoxy-2-fluoro-ß-D-arabinofuranosyl)uracil (2′-F-ANA-U) or 2′-deoxy-2′-fluoroarabinouridine. In particular, the double-stranded nucleic acid moiety comprises or consists in the following sequence (SEQ ID NO: 1):

5′CCCAGCAAACAAGCCT-∫
3′GGG [symbol] CGTTTGT [symbol] CGGA-∫

and more specifically,

5′CsCsCsAGCAAACAAGCCT-∫
3′GsGsGs [symbol] CGTTTGT [symbol] CGGA-∫

In another example, the double-stranded nucleic acid moiety or the nucleic acid of the molecule according to the present invention comprises or consists in the following sequence (SEQ ID NO: 1):

5′CCCAGCAAACAAGCCT-∫
3′ [symbol] [symbol] [symbol] TCGTTTGTTCGGA-∫

wherein the G is 2′-deoxy-2′-fluoroarabinoguanosine. In particular, the double-stranded nucleic acid moiety comprises or consists in the following sequence (SEQ ID NO: 1):

5′CCCAGCAAACAAGCCT-∫
3′ [symbol] [symbol] [symbol] TCGTTTGTTCGGA-∫

and more specifically,

5′CsCsCsAGCAAACAAGCCT-∫
3′ [symbol] [symbol] [symbol] TCGTTTGTTCGGA-∫

In another example, the double-stranded nucleic acid moiety or the nucleic acid of the molecule according to the present invention comprises or consists in the following sequence (SEQ ID NO: 1):

5′ [symbol] [symbol] [symbol] AGCAAACAAGCCT-∫
3′GGGTCGTTTGTTCGGA-∫

wherein the C is 2′-deoxy-2′-fluoroarabinocytidine. In particular, the double-stranded nucleic acid moiety comprises or consists in the following sequence (SEQ ID NO: 1):

5′ [symbol] [symbol] [symbol] AGCAAACAAGCCT-∫
3′GGGTCGTTTGTTCGGA-∫

and more specifically,

5′ [symbol] [symbol] [symbol] AGCAAACAAGCCT-∫
3′GsGsGsTCGTTTGTTCGGA-∫

Loops

The loop is linked to the 5′end of the first strand and the 3′end of the complementary strand of the double-stranded moiety, and optionally to a molecule facilitating the endocytosis.

The loop preferably comprises a chain from 10 to 100 atoms, preferably from 15 to 25 atoms.

A loop may include from 2 to 10 nucleotides, preferably, 3, 4 or 5 nucleotides. Non-nucleotide loops non exhaustively include abasic nucleotide, polyether, polyamine, polyamide, peptide, carbohydrate, lipid, polyhydrocarbon, or other polymeric compounds (e.g. oligoethylene glycols such as those having between 2 and 10 ethylene glycol units, preferably 4, 5, 6, 7 or 8 ethylene glycol units). In one embodiment, the loop can be selected from the group consisting of N-(5-hydroxymethyl-6-phosphohexyl)-11-(3-(6-phosphohexythio) succinimido)) undecamide, 1,3-bis-[5-hydroxylpentylamido]propyl-2-(6-phosphohexyl), hexaethyleneglycol, tetradeoxythymidylate (T4), 1,19-bis(phospho)-8-hydraza-2-hydroxy-4-oxa-9-oxo-nonadecane and 2,19-bis(phosphor)-8-hydraza-1-hydroxy-4-oxa-9-oxo-nonadecane.

The molecules facilitating endocytosis are conjugated to the loop, optionally through a linker. Any linker known in the art may be used to covalently attach the molecule facilitating endocytosis to the loop For instance, WO09/126933 provides a broad review of convenient linkers pages 38-45. The linker can be non-exhaustively, aliphatic chain, polyether, polyamine, polyamide, peptide, carbohydrate, lipid, polyhydrocarbon, or other polymeric compounds (e. g. oligoethylene glycols such as those having between 2 and 10 ethylene glycol units, preferably 3, 4, 5, 6, 7 or 8 ethylene glycol units, still more preferably 6 ethylene glycol units), as well as incorporating any bonds that may be break down by chemical or enzymatical way, such as a disulfide linkage, a protected disulfide linkage, an acid labile linkage (e.g., hydrazone linkage), an ester linkage, an ortho ester linkage, a phosphonamide linkage, a biocleavable peptide linkage, an azo linkage or an aldehyde linkage. Such cleavable linkers are detailed in WO2007/040469 pages 12-14, in WO2008/022309 pages 22-28.

The molecule facilitating the endocytosis is bound to the loop by any mean known by the person skilled in the art, optionally through an oligoethylene glycol spacer.

In a specific embodiment, the linker between the molecule facilitating endocytosis and the loop comprises C(O)—NH—(CH₂—CH₂—O)ₙ or NH—C(O)—(CH₂—CH₂—O)ₙ, wherein n is an integer from 1 to 10, preferably n being selected from the group consisting of 3, 4, 5 and 6. In a very particular embodiment, the linker is CO—NH—(CH₂—CH₂—O)₄ (carboxamido triethylene glycol).

In another specific embodiment, the linker between the molecule facilitating endocytosis and the loop molecule is dialkyl-disulfide {e.g., (CH₂)ₚ—S—S—(CH₂)_{q} with p and q being integer from 1 to 10, preferably from 3 to 8, for instance 61.

In another particular embodiment, the loop has been developed so as to be compatible with oligonucleotide solid phase synthesis. Accordingly, it is possible to incorporate the loop during the synthesis of the nucleic acid molecule, thereby facilitating the synthesis and reducing its cost.

The loop can have a structure selected from one of the following formulae:

—O—P(X)OH—O—{[(CH₂)₂-]_{g}—P(X)OH—O}ᵣ—K—O—P(X)OH—O—{[(CH₂)₂—O]ₕ—P(X)OH—O—}ₛ (I)

with r and s being independently an integer 0 or 1; g and h being independently an integer from 1 to 7 and the sum g+h being from 4 to 7;

with K being

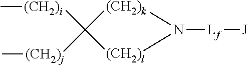

with i, j, k and 1 being independently an integer from 0 to 6, preferably from 1 to 3;

or

—O—P(X)OH—O—[(CH₂)_{d}—C(O)—NH]_{b}—CHR—[C(O)—NH—(CH₂)ₑ]_{c}—O—P(X)OH—O— (II)

with b and c being independently an integer from 0 to 4, and the sum b+c is from 3 to 7;

d and e being independently an integer from 1 to 3, preferably from 1 to 2;

with R being -L_{f}-J,

wherein X is O or S, L being a linker, preferably a linear alkylene and/or an oligoethylene glycol optionally interrupted by one or several groups selected from amino, amide, and oxo, and f being an integer being 0 or 1, and J being a molecule facilitating the endocytosis or being H.

When J is H, the molecule can be used as a synthon in order to prepare the molecule conjugated to a molecule facilitating the endocytosis. Alternatively, the molecule could also be used as a drug, without any conjugation to a molecule facilitating the endocytosis.

In a specific example, the molecule could be

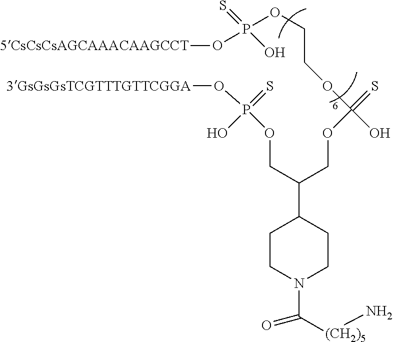

In a first aspect, the loop has a structure according to formula (I):

—O—P(X)OH—O—{[(CH₂)₂—O]_{g}—P(X)OH—O}ᵣ—K—O—P(X)OH—O—{[(CH₂)₂—O]ₕ—P(X)OH—O—}ₛ (I)

X is O or S. X can vary among O and S at each occurrence of —O—P(X)OH—O— in formula (I). Preferably, X is S.

The sum g+h is preferably from 5 to 7, especially is 6. Accordingly, if r is 0, h can be from 5 to 7 (with s being 1); if g is 1, h can be from 4 to 6 (with r and s being 1); if g is 2, h can be from 3 to 5 (with r and s being 1); if g is 3, h can be from 2 to 4 (with r and s being 1); if g is 4, h can be from 1 to 3 (with r and s being 1); if g is 5, h can be from 1 to 2 (with r being 1 and s being 0 or 1); or if g is 6 or 7, s is 0 (with r being 1).

Preferably, i and j can be the same integer or can be different. i and j can be selected from the integer 1, 2, 3, 4, 5 or 6, preferable 1, 2 or 3, still more particularly 1 or 2, especially 1.

Preferably, k and 1 are the same integer. In one aspect, k and 1 are an integer selected from 1, 2 or 3, preferably 1 or 2, more preferably 2.

Accordingly, K can be

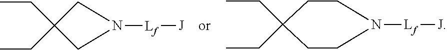

In a preferred aspect, K is

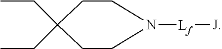

In on specific aspect, the loop has the formula (I)

—O—P(X)OH—O—{[(CH₂)₂—O]_{g}—P(X)OH—O}ᵣ—K—O—P(X)OH—O—{[(CH₂)₂—O]ₕ—P(X)OH—O—}ₛ (I)

with X being S, r being 1, g being 6, s being 0, and K being

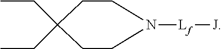

In a particular aspect, f is 1 and L-J is —C(O)—(CH₂)ₘ—NH—[C(O)]ₜ—[(CH₂)₂—O]ₙ—(CH₂)ₚ—[C(O)]ₙ-J or —C(O)—(CH₂)ₘ—NH—[C(O)—CH₂—O]ₜ—[(CH₂)₂—O]ₙ—(CH₂)ₚ—[C(O)]ᵥ-J with m being an integer from 0 to 10; n being an integer from 0 to 15; p being an integer from 0 to 4; t and v being an integer 0 or 1 with at least one among t and v being 1.

More particularly, f is 1 and L-J is selected in the group consisting of —C(O)—(CH₂)ₘ NH—[(CH₂)₂—O]ₙ—(CH₂)ₚ—C(O)-J, —C(O)—(CH₂)ₘ—NH—C(O)—[(CH₂)₂—O]ₙ—(CH₂)ₚ-J, C(O)—(CH₂)ₘ—NH—C(O)—CH₂—O—[(CH₂)₂—O]ₙ—(CH₂)ₚ-J, —C(O)—(CH₂)ₘ—NH—C(O)—[(CH₂)₂—O]ₙ—(CH₂)ₚ—C(O)-J and —C(O)—(CH₂)ₘ—NH—C(O)—CH₂—O—[(CH₂)₂—O]ₙ—(CH₂)ₚ—C(O)-J, with m being an integer from 0 to 10; n being an integer from 0 to 15; and p being an integer from 0 to 3.

Optionally, f is 1 and L-J is selected in the group consisting of —C(O)—(CH₂)₅—NH—[(CH₂)₂—O]₃₋₁₃—CH₂—C(O)-J, —C(O)—(CH₂)₅—NH—C(O)—[(CH₂)₂—O]₃₋₁₃—CH₂-J, C(O)—(CH₂)₅—NH—C(O)—CH₂—O—[(CH₂)₂—O]₃₋₁₃—CH₂-J, —C(O)—(CH₂)₅—NH—C(O)—[(CH₂)₂—O]₃₋₁₃—CH₂—C(O)-J and —C(O)—(CH₂)₅—NH—C(O)—CH₂—O—[(CH₂)₂—O]₃₋₁₃—CH₂—C(O)-J or —C(O)—(CH₂)₅—NH—C(O)-J.

For instance, f can be 1 and L-J is selected from the group consisting of —C(O)—(CH₂)₅—NH—[(CH₂)₂—O]₃—(CH₂)₂—C(O)-J, —C(O)—(CH₂)₅—NH—C(O)—[(CH₂)₂—O]₃—(CH₂)₃-J, —C(O)—(CH₂)₅—NH—C(O)—CH₂—O—[(CH₂)₂—O]₅—CH₂—C(O)-J, —C(O)—(CH₂)₅—NH—C(O)—CH₂—O—[(CH₂)₂—O]₉—CH₂—C(O)-J, —C(O)—(CH₂)₅—NH—C(O)—CH₂—O—[(CH₂)₂—O]₁₃—CH₂—C(O)-J, or —C(O)—(CH₂)₅—NH—C(O)-J.

In a very particular aspect, f is 1 and L-J is —C(O)—(CH₂)ₘ—NH—[(CH₂)₂—O]ₙ—(CH₂)ₚ—C(O)-J with m being an integer from 0 to 10, preferably from 4 to 6, especially 5; n being an integer from 0 to 6; and p being an integer from 0 to 2. In a particular aspect, m is 5 and, n and p are 0. In another particular aspect, m is 5, n is 3 and p is 2.

In a second aspect of the disclosure, the loop has a structure according to formula (II):

—O—P(X)OH—O—[(CH₂)_{d}—C(O)—NH]_{b}—CHR—[C(O)—NH—(CH₂)ₑ]_{c}—O—P(X)OH—O— (II)

with X being O or S;

b and c being independently an integer from 0 to 4, and the sum b+c is from 3 to 7;

d and e being independently an integer from 1 to 3, preferably from 1 to 2;

with R being —(CH₂)₁₋₅—C(O)—NH-L_{f}-J or —(CH₂)₁₋₅—NH—C(O)-L_{f}-J, and

with L being a linker, preferably a linear alkylene or an oligoethylene glycol, f being an integer being 0 or 1, and J being a molecule facilitating the endocytosis.

If b and/or c are 2 or more, d and e can be different in each occurrence of [(CH₂)_{d}—C(O)—NH] or —[C(O)—NH—(CH₂)ₑ].

In one aspect, when d and e are 2, the sum b+c is from 3 to 5, in particular 4. For instance, b can be 0 and c is from 3 to 5; b can be 1 and c is from 2 to 4; b can be 2 and c is from 1 to 3; or b can be from 3 to 5 and c is 0.

In one aspect, when d and e are 1, the sum b+c is from 4 to 7, in particular 5 or 6. For instance, b can be 0 and c is from 3 to 6; b can be 1 and c is from 2 to 5; b can be 2 and c is from 1 to 4; or b can be from 3 to 6 and c is 0.

In one aspect, b, c, d and e are selected so as the loop comprises a chain from 10 to 100 atoms, preferably from 15 to 25 atoms.

In a non-exhaustive list of examples, the loop could be one of the followings:
—O—P(X)OH—O—(CH₂)₂—C(O)—NH—(CH₂)₂—C(O)—NH—CHR—C(O)—NH—(CH₂)₂—C(O)—NH—(CH₂)₂—O—P(X)OH—O—
—O—P(X)OH—O—(CH₂)₂—C(O)—NH—CHR—C(O)—NH—(CH₂)₂—C(O)—NH—(CH₂)₂—C(O)—NH—(CH₂)₂—O—P(X)OH—O—
—O—P(X)OH—O—CHR—C(O)—NH—(CH₂)₂—C(O)—NH—(CH₂)₂—C(O)—NH—(CH₂)₂—C(O)—NH—(CH₂)₂—O—P(X)OH—O—
—O—P(X)OH—O—(CH₂)₂—C(O)—NH—(CH₂)₂—C(O)—NH—(CH₂)₂—C(O)—NH—CHR—C(O)—NH—(CH₂)₂—O—P(X)OH—O—
—O—P(X)OH—O—(CH₂)₂—C(O)—NH—(CH₂)₂—C(O)—NH—(CH₂)₂—C(O)—NH—(CH₂)₂—C(O)—NH—CHR—O—P(X)OH—O—
—O—P(X)OH—O—(CH₂)₂—C(O)—NH—(CH₂)—C(O)—NH—CHR—C(O)—NH—(CH₂)—C(O)—NH—(CH₂)₂—O—P(X)OH—O—
—O—P(X)OH—O—(CH₂)—C(O)—NH—(CH₂)₂—C(O)—NH—CHR—C(O)—NH—(CH₂)₂—C(O)—NH—(CH₂)—O—P(X)OH—O—, or
—O—P(X)OH—O—(CH₂)—C(O)—NH—(CH₂)—C(O)—NH—CHR—C(O)—NH—(CH₂)—C(O)—NH—(CH₂)—O—P(X)OH—O—

In a particular aspect, the loop can be the following:
—O—P(X)OH—O—(CH₂)₂—C(O)—NH—(CH₂)₂—C(O)—NH—CHR—C(O)—NH—(CH₂)₂—C(O)—NH—(CH₂)₂—O—P(X)OH—O—

with R being -L_{f}-J; and

with L being a linker, preferably a linear alkylene and/or an oligoethylene glycol optionally interrupted by one or several groups selected from amino, amide, and oxo, and f being an integer being 0 or 1.

Preferably, X is S.

L can be —(CH₂)₁₋₅—C(O)-J, preferably —CH₂—C(O)-J or —(CH₂)₂—C(O)-J.

Alternatively, L-J can be —(CH₂)₄—NH—[(CH₂)₂—O]ₙ—(CH₂)ₚ—C(O)-J with n being an integer from 0 to 6; and p being an integer from 0 to 2. In a particular aspect, n is 3 and p is 2.

Molecules Facilitating Endocytosis

The nucleic acid molecules of the present invention are optionally conjugated to a molecule facilitating endocytosis, referred as J in the above formulae. Therefore, in a first aspect, J is a molecule facilitating endocytosis. In an alternative aspect, J is a hydrogen.

The molecules facilitating endocytosis may be lipophilic molecules such as cholesterol, single or double chain fatty acids, or ligands which target cell receptors enabling receptor mediated endocytosis, such as folic acid and folate derivatives or transferrin (Goldstein et al. Ann. Rev. Cell Biol. 1985 1:1-39; Leamon & Lowe, Proc Natl Acad Sci USA. 1991, 88: 5572-5576.). Fatty acids may be saturated or unsaturated and be in C₄-C₂₈, preferably in C₁₄-C₂₂, still more preferably being in C₁₈ such as oleic acid or stearic acid. In particular, fatty acids may be octadecyl or dioleoyl. Fatty acids may be found as double chain form linked with an appropriate linker such as a glycerol, a phosphatidylcholine or ethanolamine and the like or linked together by the linkers used to attach on the conjugated nucleic acid molecule. As used herein, the term “folate” is meant to refer to folate and folate derivatives, including pteroic acid derivatives and analogs. The analogs and derivatives of folic acid suitable for use in the present invention include, but are not limited to, antifolates, dihydrofolates, tetrahydrofolates, folinic acid, pteropolyglutamic acid, 1-deaza, 3-deaza, 5-deaza, 8-deaza, 10-deaza, 1,5-deaza, 5,10-dideaza, 8,10-dideaza, and 5,8-dideaza folates, antifolates, and pteroic acid derivatives. Additional folate analogs are described in US2004/242582.

Accordingly, the molecule facilitating endocytosis may be selected from the group consisting of single or double chain fatty acids, folates and cholesterol. More preferably, the molecule facilitating endocytosis is selected from the group consisting of dioleoyl, octadecyl, folic acid, and cholesterol. In a most preferred embodiment, the molecule facilitating endocytosis is a cholesterol.

Accordingly, in one preferred embodiment, the conjugated nucleic acid molecule (also referred as OX401) has the following formula:

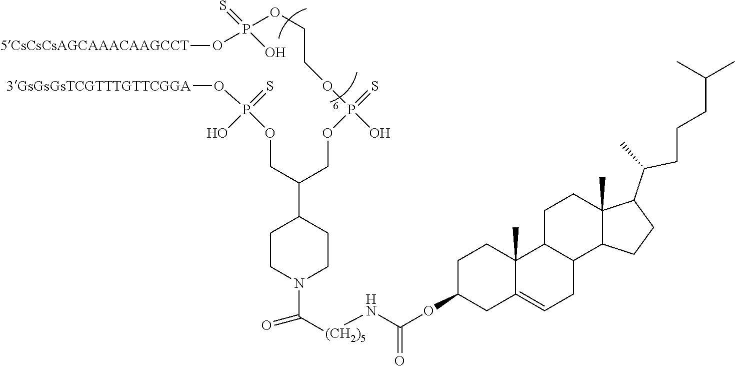

wherein internucleotide linkages “s” refers to phosphorothioate internucleotide linkages.

In another specific embodiment, the conjugated nucleic acid molecule (also referred as OX402) has the following formula:

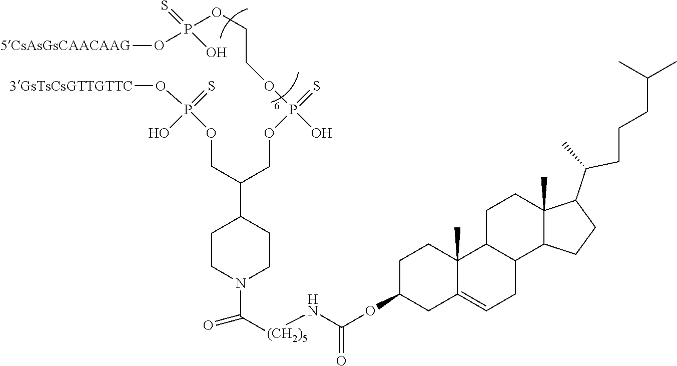

wherein internucleotide linkages “s” refers to phosphorothioate internucleotide linkages.

In still another preferred embodiment, the conjugated nucleic acid molecule has the following formula:

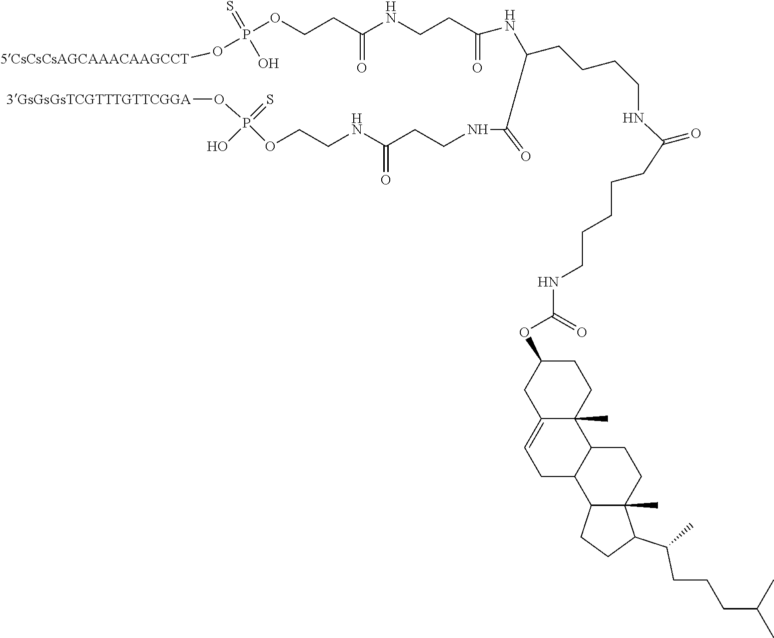

wherein internucleotide linkages “s” refers to phosphorothioate internucleotide linkages.

In other preferred embodiments, the conjugated nucleic acid molecule has any of the following formulae:

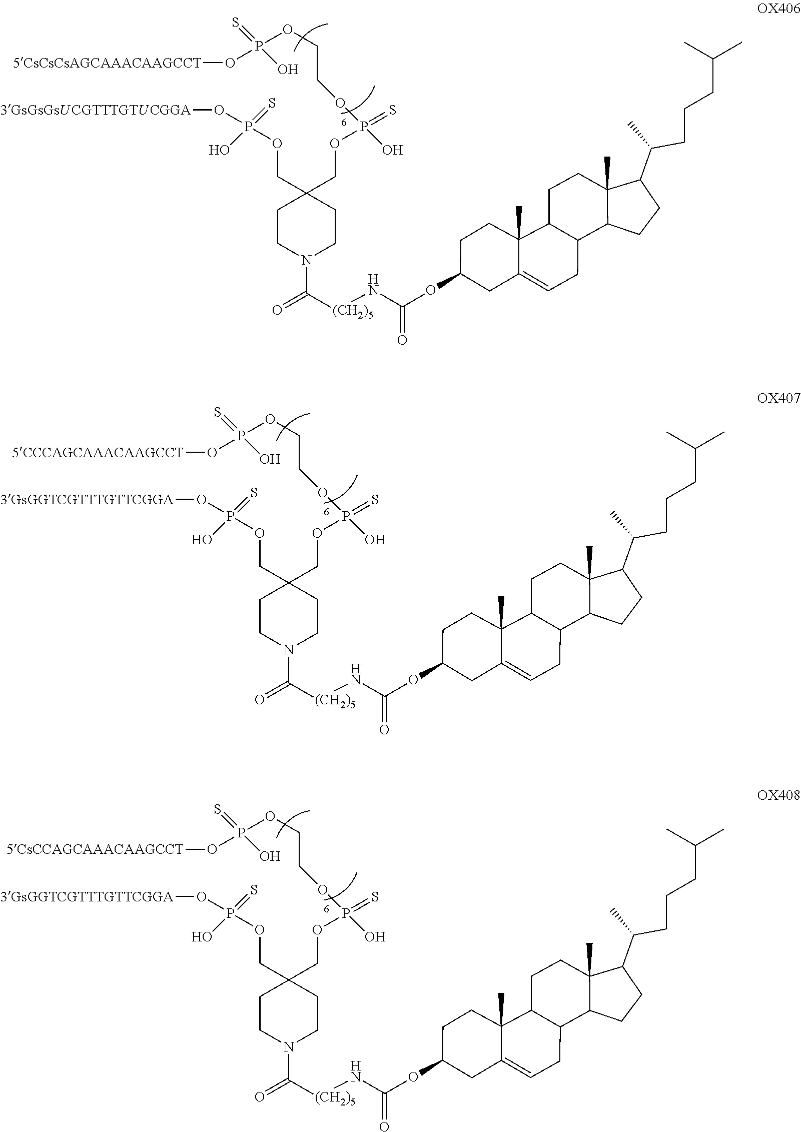

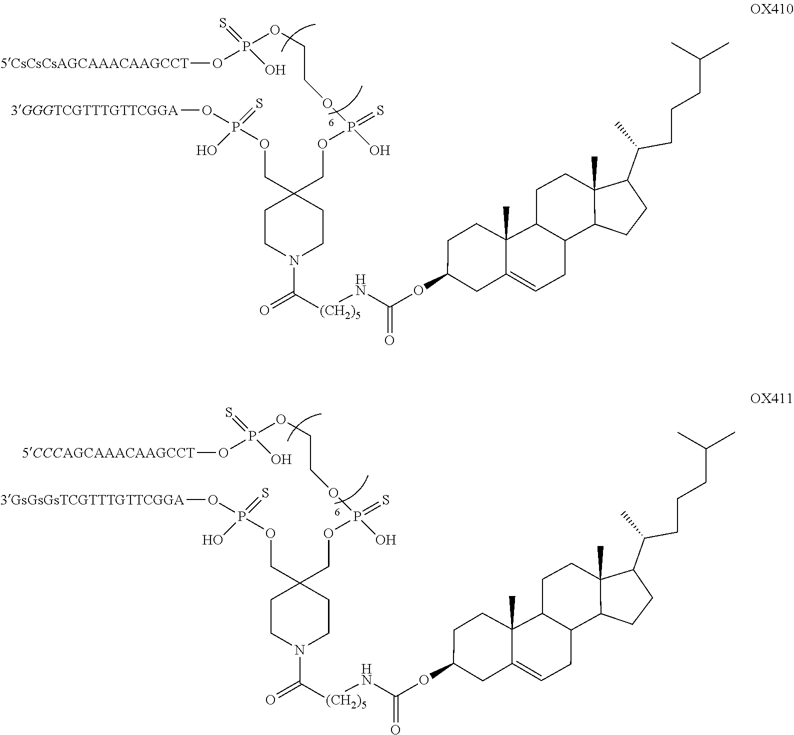

wherein internucleotide linkages “s” refers to phosphorothioate internucleotide linkages; italic U being 2′-deoxy-2′-fluoroarabinouridine, italic G being 2′-deoxy-2′-fluoroarabinoguanosine; italic C being 2′-deoxy-2′-fluoroarabinocytidine.

Alternatively, the molecule facilitating endocytosis may also be tocopherol, sugar such as galactose and mannose and their oligosaccharide, peptide such as RGD and bombesin, and proteins such as integrin.

Sigma-2 Receptor Ligands

In a particular aspect, the molecule facilitating endocytosis is selected in order to target cancer cells. Then, it is chosen so as to be a ligand of a receptor which is specifically expressed in cancer cells or is overexpressed in cancer cells in comparison with normal cells.

In this context, the molecule facilitating endocytosis can be a ligand of a sigma-2 receptor (σ2R).

The term “sigma-2 receptor (σ2R)” refers to a sigma receptor subtype that has been found highly expressed in malignant cancer cells (e.g. breast, ovarian, lung, brain, bladder, colon, and melanoma). The sigma-2 receptor is a cytochrome related protein located in the lipid raft that is most commonly associated with P450 proteins, and is coupled with the PGRMC1 complex, EGFR, mTOR, caspases, and various ion channels.

The term “sigma-2 receptor (σ2R) ligand” refers to an agonist compound synthetic or not which binds with high selectivity and affinity to σ2R, and is then internalized by endocytosis. σ2R agonists inhibit tumor cell proliferation and induce apoptosis in cancer cells.

In one preferred aspect, the sigma-2 receptor (σ2R) ligand is a azabicyclononane analog, more particularly a N-substituted-9-azabicyclo[3.3.1]nonan-3α-yl carbamate analog as described in Vangveravong et al. Bioorg. Med. Chem (2006) comprising the following formula:

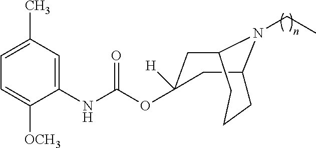

in particular

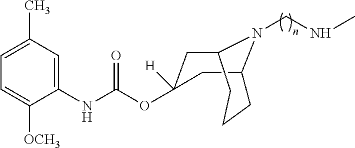

wherein n is an integer from 1 to 20. Optionally, n is an integer from 1 to 10, from 2 to 9, from 3 to 8, from 4 to 7 or from 5 to 6.

In a first par aspect, the 62R ligand has the following formula

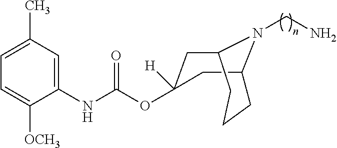

wherein n is an integer from 1 to 20. Optionally, n is an integer from 1 to 10, from 2 to 9, from 3 to 8, from 4 to 7 or from 5 to 6.

In a particular embodiment, the σ2R ligand is referred as SV119 (n=6) and has the following formula:

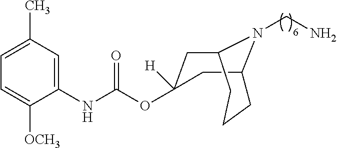

In still another particular embodiment, the σ2R ligand is referred as SW43 (n=10) and has the following formula:

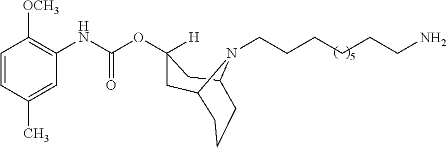

In another embodiment, the σ2R ligand is a N-substituted-9-azabicyclo[3.3.1]nonan-3α-yl carbamate analog and has the following formula:

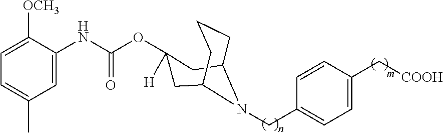

wherein n is an integer from 1 to 20 and m is an integer from 0 to 10.

In a particular embodiment, the σ2R ligand has the following formula:

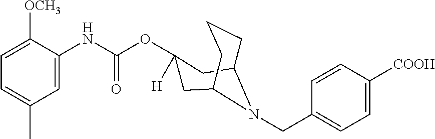 [SMILES: COc1ccc(C)cc1NC(=O)OC1CC2CCCC(C1)N2Cc1ccc(C(=O)O)cc1]

The σ2R ligand is conjugated to the nucleic acid molecule through the loop by the carboxy or amino group, optionally via a linker.

Accordingly, in one preferred embodiment, the conjugated nucleic acid molecule has the following formula:

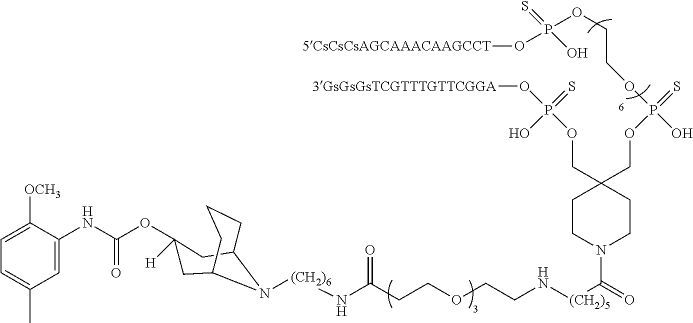

wherein internucleotide linkages “s” refers to phosphorothioate internucleotide linkages.

Accordingly, in still another preferred embodiment, the conjugated nucleic acid molecule (also referred as OX405) has the following formula:

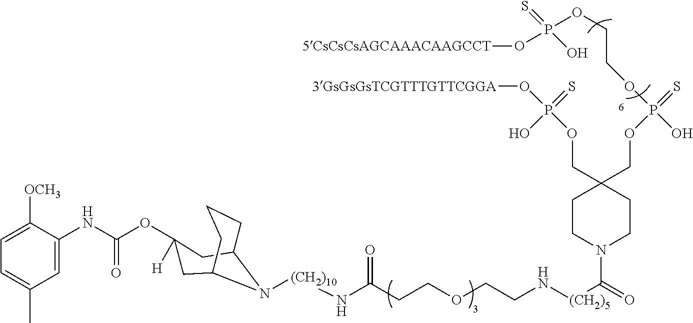

wherein internucleotide linkages “s” refers to phosphorothioate internucleotide linkages.

Accordingly, in still another preferred embodiment, the conjugated nucleic acid molecule (also referred as OX405) has the following formula:

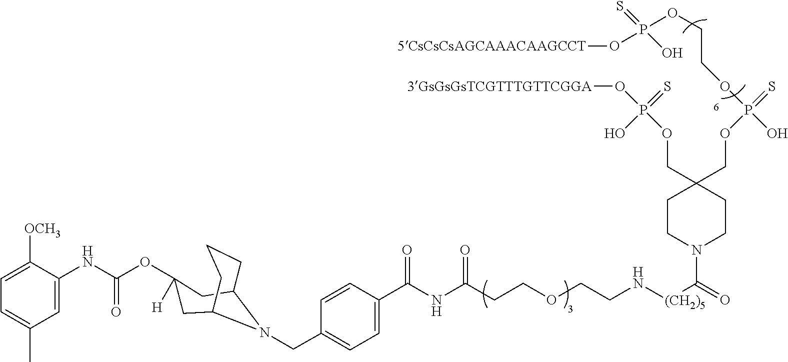

wherein internucleotide linkages “s” refers to phosphorothioate internucleotide linkages.

In still another preferred embodiment, the conjugated nucleic acid molecule (also referred as OX405) has the following formula:

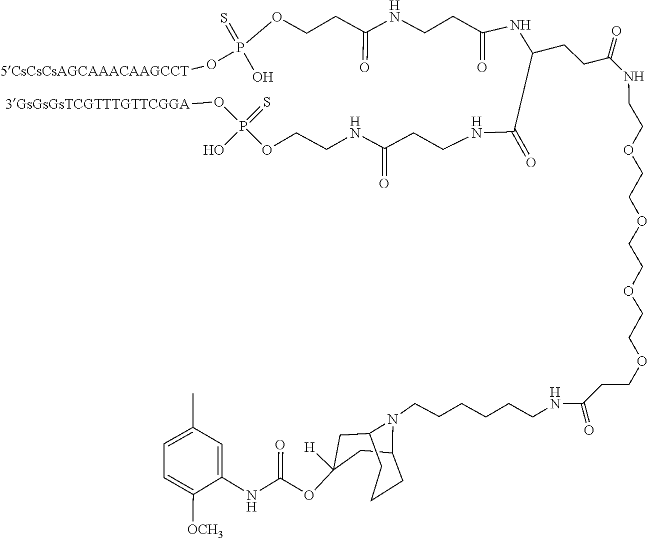

wherein internucleotide linkages “s” refers to phosphorothioate internucleotide linkages.

In other preferred embodiments, the conjugated nucleic acid molecule has any of the following formulae:

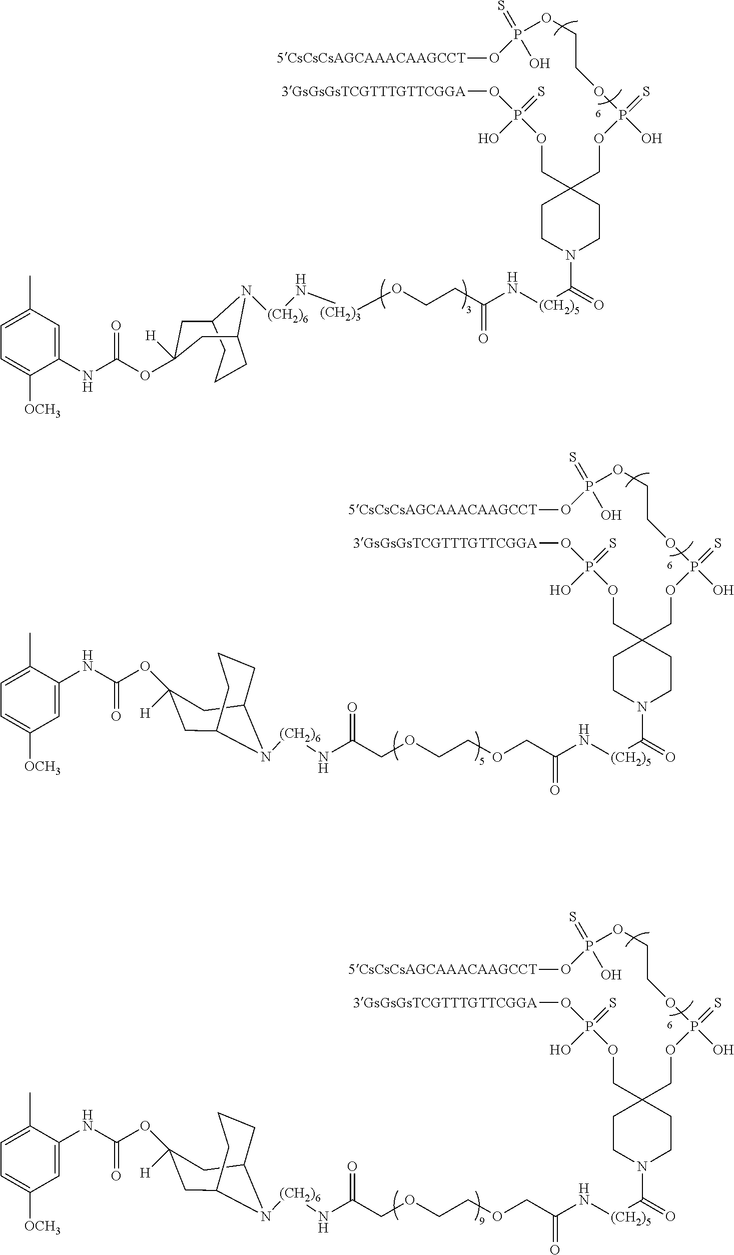

the above compound being also referred as OX403

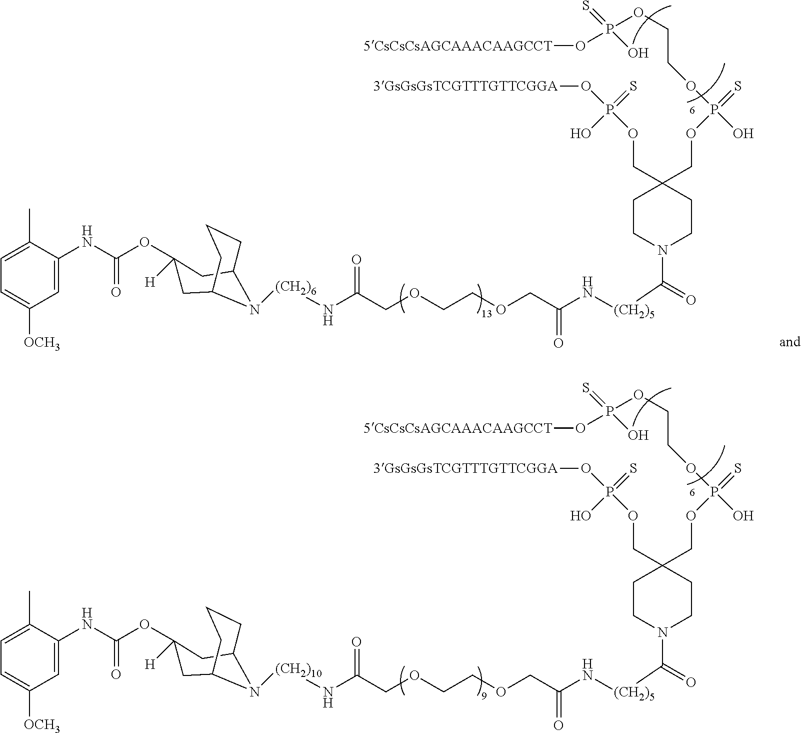

the above compound being also referred as OX404
wherein internucleotide linkages “s” refers to phosphorothioate internucleotide linkages.

Therapeutic Uses of the Nucleic Acid Molecules

The conjugated nucleic acid molecules according to the present invention are able to active PARP. They lead to an increase of micronuclei and cytotoxicity in cancer cells. They show specificity toward cancer cells which may preclude or limit side effects. In addition, the specific increase of micronuclei in cancer cells leads to an early activation of the STING pathway.

Accordingly, the conjugated nucleic acid molecules according to the present invention can be used as a drug, especially for the treatment of cancer.

Therefore, the present invention relates to a conjugated nucleic acid molecule according to the present invention for use as a drug. It further relates to a pharmaceutical composition comprising a conjugated nucleic acid molecule according to the present invention, especially for use for the treatment of cancer.

The pharmaceutical compositions contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parental administration, the active compounds(s) may be formulated in a unit dosage form for injection in vehicles such as saline, dextrose solution, serum albumin and Ringer's solution.

The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parental administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavouring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

The pharmaceutical compositions and the products, kits or combined preparation described in the invention can be used for treating cancer in a subject.

The terms “cancer” and “cancerous” refer to or describe the physiological condition in mammals that is typically characterized by unregulated cell growth. Examples of cancer include, but are not limited to, solid tumors and hematological cancers, including carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric cancer including gastrointestinal cancer, pancreatic cancer, glioblastoma, neuroblastoma, cervical cancer, ovarian cancer, liver cancer, bladder cancer, urinary tract cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. Additional cancer indications are disclosed herein.

In a particular embodiment, “cancer” refers to tumor cells carrying NAD⁺ depletion, for instance selected from ERCC1 or ATM deficiency or cancer cells carrying IDHs mutations.

In very particular embodiment, a clinical stratification or a selection of better responders is possible for patients with tumors showing deficiencies in the NAD⁺ synthesis, in particular for patients with tumors carrying NAD⁺ depletion.

Determining the optimal dosage will generally involve the balancing of the level of therapeutic benefit against any risk or deleterious side effects of the treatments of the present invention. The selected dosage level will depend on a variety of factors including, but not limited to, the activity of the conjugated nucleic acid molecule, the route of administration, the time of administration, the rate of excretion of the compound, the duration of the treatment, other drugs, compounds, and/or materials used in combination, and the age, sex, weight, condition, general health, and prior medical history of the patient. The amount of conjugated nucleic acid molecule and route of administration will ultimately be at the discretion of the physician, although generally the dosage will be to achieve local concentrations at the site of action which achieve the desired effect.

The administration route for the conjugated nucleic acid molecule as disclosed herein may be oral, parental, intravenous, intratumoral, subcutaneous, intracranial, intra-arterial, topical, rectal, transdermal, intradermal, nasal, intramuscular, intraperitoneal, intraosseous, and the like. In a preferred embodiment, the conjugated nucleic acid molecules are to be administered or injected near the tumoral site(s) to be treated.

For instance, the efficient amount of the conjugated nucleic acid molecules be from 0.01 to 1000 mg, for instance preferably from 0.1 to 100 mg. Of course, the dosage and the regimen can be adapted by the one skilled in the art in consideration of the chemotherapy and/or radiotherapy regimen.

The conjugated nucleic acid molecule according to the present invention can be used in combination with an additional therapeutic agent. The additional therapeutic agent can be for instance an immunomodulatory such as an immune checkpoint inhibitor, a T-cell-based cancer immunotherapy including adoptive cell transfer (ACT), genetically modified T-cells or engineered T-cells such as chimeric antigen receptor cells (CAR-T cells), a conventional chemotherapeutic, radiotherapeutic or anti-angiogenic agent, HDAC inhibitor (such as belinostat) or targeted immunotoxin.

Combinations with Immunomodulators/Immune Checkpoint Inhibitors (ICI)

The inventors demonstrated the high antitumor therapeutic efficiency of the combination of a conjugated nucleic acid molecule with an immunomodulator such as an immune checkpoint inhibitor (ICI), preferably an inhibitor of the PD-1/PD-L1 pathway, as suggested by the activation of the STING pathway and the increase of the PD-L1 expression. The invention thus provides combined therapies in which a conjugated nucleic acid molecule of the invention is administered to patients with, before, or after an immunomodulator such as an immune checkpoint inhibitor (ICI).

Accordingly, the present invention concerns a pharmaceutical composition comprising a conjugated nucleic acid molecule of the invention and an immunomodulator, more particularly for use in the treatment of cancer. The present invention also concerns a product comprising a conjugated nucleic acid molecule of the invention and an immunomodulator as a combined preparation for simultaneous, separate or sequential use, more particularly for use in the treatment of cancer. In a preferred embodiment, the immunomodulator is an inhibitor of the PD-1/PD-L1 pathway.

The invention also provides a method of treating cancer by administering to a patient in need thereof a conjugated nucleic acid molecule of the present invention in combination with one or more immunomodulators (e.g., one or more of an activator of a costimulatory molecule or an inhibitor of an immune checkpoint molecule). In a preferred embodiment, the immunomodulator is an inhibitor of the PD-1/PD-L1 pathway.

Activator of a Costimulatory Molecule:

In certain embodiments, the immunomodulator is an activator of a costimulatory molecule. In one embodiment, the agonist of the costimulatory molecule is selected from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1 BB (CD137), GITR, CD30, CD40, BAFFR, HVEM, CD7, LIGHT, NKG2C, SLAMF7, NKp80, CD160, B7-H3 or CD83 ligand.

### Inhibitor of an Immune Checkpoint Molecule:

In certain embodiments, the immunomodulator is an inhibitor of an immune checkpoint molecule. In one embodiment, the immunomodulator is an inhibitor of PD-1, PD-L1, PD-L2, CTLA-4, TIM-3, LAG-3, NKG2D, NKG2L, KIR, VISTA, BTLA, TIGIT, LAIR1, CD160, 2B4 and/or TGFRbeta. In one embodiment, the inhibitor of an immune checkpoint molecule inhibits PD-1, PD-L1, LAG-3, TIM-3 or CTLA-4, or any combination thereof. The term “inhibition” or “inhibitor” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40%, 50% or more is included by this term. Thus, inhibition need not be 100%.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1 Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA-4, TIM-3, LAG-3, NKG2D, NKG2L, KIR VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) selected from, e.g., the heavy chain constant regions of IgG1, IgG2, IgG3, IgG4, IgM, IgA1, IgA2, IgD, and IgE; particularly, selected from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of Fc receptor binding, antibody glycosylation, the number of cysteine residues, effector cell function, or complement function). In certain embodiments, the antibody molecule is in the form of a bispecific or multispecific antibody molecule.

PD-1 Inhibitors

In some embodiments, the conjugated nucleic acid molecule of the present invention is administered in combination with a PD-1 inhibitor. In some embodiments, the PD-1 inhibitor is selected from PDR001 (Novartis), Nivolumab (Bristol-Myers Squibb), Pembrolizumab (Merck & Co), Pidilizumab (CureTech), MEDIO680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

Exemplary PD-1 Inhibitors

In some embodiments, the anti-PD-1 antibody is Nivolumab (CAS Registry Number: 946414-94-4). Alternative names for Nivolumab include MDX-1106, MDX-1106-04, ONO— 4538, BMS-936558 or OPDIVO®. Nivolumab is a fully human lgG4 monoclonal antibody which specifically blocks PD1. Nivolumab (clone 5C4) and other human monoclonal antibodies that specifically bind to PD1 are disclosed in U.S. Pat. No. 8,008,449 and PCT Publication No. WO 2006/121168, which are incorporated herein by reference in their entirety.

In other embodiments, the anti-PD-1 antibody is Pembrolizumab. Pembrolizumab (Trade name KEYTRUDA formerly Lambrolizumab, also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, PCT Publication No. WO 2009/114335, and U.S. Pat. No. 8,354,509, which are incorporated herein by reference in their entirety.

In some embodiments, the anti-PD-1 antibody is Pidilizumab. Pidilizumab (CT-011; CureTech) is a humanized lgG1 k monoclonal antibody that binds to PD1. Pidilizumab and other humanized anti-PD-1 monoclonal antibodies are disclosed in PCT Publication No. WO 2009/101611, which are incorporated herein by reference in their entirety.

Other anti-PD1 antibodies are disclosed in U.S. Pat. No. 8,609,089, US Publication No. 2010028330, and/or US Publication No. 20120114649, which are incorporated herein by reference in their entirety. Other anti-PD1 antibodies include AMP514 (Amplimmune).

In one embodiment, the anti-PD-1 antibody molecule is MEDIO680 (Medimmune), also known as AMP-514. MEDI0680 and other anti-PD-1 antibodies are disclosed in U.S. Pat. No. 9,205,148 and WO 2012/145493, which are incorporated herein by reference in their entirety.

In one embodiment, the anti-PD-1 antibody molecule is REGN2810 (Regeneron).

In one embodiment, the anti-PD-1 antibody molecule is PF-06801591 (Pfizer).

In one embodiment, the anti-PD-1 antibody molecule is BGB-A317 or BGB-108 (Beigene).

In one embodiment, the anti-PD-1 antibody molecule is INCSHR1210 (Incyte), also known as INCSHR01210 or SHR-1210.

In one embodiment, the anti-PD-1 antibody molecule is TSR-042 (Tesaro), also known as ANB011.

Further known anti-PD-1 antibodies include those described, e.g., in WO 2015/1 12800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, which are incorporated herein by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, which is incorporated herein by reference in its entirety. In some embodiments, the PD-1 inhibitor is an immunoadhesin {e.g., an immunoadhesin comprising an extracellular or PD-1 binding portion of PD-L1 or PD-L2 fused to a constant region (e.g., an Fc region of an immunoglobulin sequence)}. In some embodiments, the PD-1 inhibitor is AMP-224 (B7-DCIg (Amplimmune), e.g., disclosed in WO 2010/027827 and WO 2011/066342, which are incorporated herein by reference in their entirety.

PD-L1 Inhibitors

In certain embodiments, the inhibitor of an immune checkpoint molecule is an inhibitor of PD-L1. In some embodiments, the conjugated nucleic acid molecule of the present invention is administered in combination with a PD-L1 inhibitor. In some embodiments, the PD-L1 inhibitor is selected from FAZ053 (Novartis), Atezolizumab (Genentech/Roche), Avelumab (Merck Serono and Pfizer), Durvalumab (Medlmmune/AstraZeneca), or BMS-936559 (Bristol-Myers Squibb).

Exemplary PD-L1 Inhibitors

In one embodiment, the PD-L1 inhibitor is an anti-PD-L1 antibody molecule. In one embodiment, the anti-PD-L1 antibody molecule is Avelumab (Merck Serono and Pfizer), also known as MSB0010718C. Avelumab and other anti-PD-L1 antibodies are disclosed in WO 2013/079174, which is incorporated herein by reference in its entirety.

In one embodiment, the anti-PD-L1 antibody molecule is Durvalumab (Medlmmune/AstraZeneca), also known as MEDI4736. Durvalumab and other anti-PD-L1 antibodies are disclosed in U.S. Pat. No. 8,779,108, which is incorporated herein by reference in its entirety.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, which are incorporated herein by reference in their entirety.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, which are incorporated herein by reference in their entirety.

In one embodiment, the anti-PD-L1 antibody is an antibody that competes for binding with, and/or binds to the same epitope on PD-L1 as, one of the anti-PD-L1 antibodies described herein.

LAG-3 Inhibitors

In certain embodiments, the inhibitor of an immune checkpoint molecule is an inhibitor of LAG-3. In some embodiments, the conjugated nucleic acid molecule of the present invention is administered in combination with a LAG-3 inhibitor. In some embodiments, the LAG-3 inhibitor is selected from LAG525 (Novartis), BMS-986016 (Bristol-Myers Squibb), or TSR-033 (Tesaro).

Exemplary LAG-3 Inhibitors

In one embodiment, the LAG-3 inhibitor is an anti-LAG-3 antibody molecule. In one embodiment, the LAG-3 inhibitor is BMS-986016 (Bristol-Myers Squibb), also known as BMS986016. BMS-986016 and other anti-LAG-3 antibodies are disclosed in WO 2015/116539 and U.S. Pat. No. 9,505,839, which are incorporated herein by reference in their entirety.

In one embodiment, the anti-LAG-3 antibody molecule is TSR-033 (Tesaro).

In one embodiment, the anti-LAG-3 antibody molecule is IMP731 or GSK2831781 (GSK and Prima BioMed). IMP731 and other anti-LAG-3 antibodies are disclosed in WO2008/132601 and U.S. Pat. No. 9,244,059, which are incorporated herein by reference in their entirety.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, which are incorporated herein by reference in their entirety.

TIM-3 Inhibitors

In certain embodiments, the inhibitor of an immune checkpoint molecule is an inhibitor of TIM-3. In some embodiments, the conjugated nucleic acid molecule of the present invention is administered in combination with a TIM-3 inhibitor. In some embodiments, the TIM-3 inhibitor is MGB453 (Novartis) or TSR-022 (Tesaro).

Exemplary TIM-3 Inhibitors

In one embodiment, the anti-TIM-3 antibody molecule is TSR-022 (AnaptysBio/Tesaro).

In one embodiment, the anti-TIM-3 antibody is APE5137 or APE5121. APE5137, APE512, and other anti-TIM-3 antibodies are disclosed in WO 2016/161270, which is incorporated herein by reference in its entirety.

Further known anti-TIM-3 antibodies include those described, e.g., in WO 2016/111947, WO 2016/071448, WO 2016/144803, U.S. Pat. Nos. 8,552,156, 8,841,418, and 9,163,087, which are incorporated herein by reference in their entirety.

NKG2D Inhibitors

In certain embodiments, the inhibitor of the NKG2D/NKG2DL pathway is an inhibitor of NKG2D. In some embodiments, the conjugated nucleic acid molecule of the present invention is administered in combination with a NKG2D inhibitor. In some embodiments, the NKG2D inhibitor is an anti-NKG2D antibody molecule such as the anti-NKG2D antibody NNC0142-0002 (also known as NN 8555, IPH2301 or JNJ-4500).

Exemplary NKG2D Inhibitors

In one embodiment, the anti-NKG2D antibody molecule is NNCO142-0002 (Novo Nordisk) as disclosed in WO 2009/077483 and U.S. Pat. No. 7,879,985, which are incorporated herein by reference in its entirety.

In another embodiment, the anti-NKG2D antibody molecule is JNJ-64304500 (Janssen) as disclosed in WO 2018/035330, which is incorporated herein by reference in its entirety.

In some embodiments, the anti-NKG2D antibodies are the human monoclonal antibodies 16F16, 16F31, MS, and 21F2 produced, isolated, and structurally and functionally characterized as described in U.S. Pat. No. 7,879,985. Further known anti-NKG2D antibodies include those described, e.g., in WO 2009/077483, WO 2010/017103, WO 2017/081190, WO 2018/035330 and WO 2018/148447, which are incorporated herein by reference in its entirety.

In some other embodiments, the NKG2D inhibitor is an immunoadhesin {e.g., an immunoadhesin comprising an extracellular or NKG2D binding portion of NKG2DL fused to a constant region (e.g., an Fc region of an immunoglobulin sequence as disclosed in WO 2010/080124, WO 2017/083545 and WO 2017/083612, which are incorporated herein by reference in its entirety).

NKG2DL Inhibitors

In some embodiments, the inhibitor of the NKG2D/NKG2DL pathway is an inhibitor of NKG2DL such as MICA, MICB, ULBP1, ULBP2, ULBP3, ULBP4, or a member of the RAET1 family. In some embodiments, the conjugated nucleic acid molecule of the present invention is administered in combination with a NKG2DL inhibitor. In some embodiments, the NKG2DL inhibitor is an anti-NKG2DL antibody molecule such as an anti-MICA/B antibody.

Exemplary MICA/MICB Inhibitors

In one embodiment, the anti-MICA/B antibody molecule is IPH4301 (Innate Pharma) as disclosed in WO 2017/157895, which is incorporated herein by reference in its entirety.

Further known anti-MICA/B antibodies include those described, e.g., in WO 2014/140904 and WO 2018/073648, which are incorporated herein by reference in its entirety.

KIR Inhibitors

In certain embodiments, the inhibitor of an immune checkpoint molecule is an inhibitor of KIR. In some embodiments, the conjugated nucleic acid molecule of the present invention is administered in combination with a KIR inhibitor. In some embodiments, the KIR inhibitor is Lirilumab (also previously referred to as BMS-986015 or IPH2102).

Exemplary KIR Inhibitors

In one embodiment, the anti-KIR antibody molecule is Lirilumab (Innate Pharma/AstraZeneca) as disclosed in WO 2008/084106 and WO 2014/055648, which are incorporated herein by reference in their entirety.

Further known anti-KIR antibodies include those described, e.g., in WO 2005/003168, WO 2005/009465, WO 2006/072625, WO 2006/072626, WO 2007/042573, WO 2008/084106, WO 2010/065939, WO 2012/071411 and WO/2012/160448, which are incorporated herein by reference in their entirety.

Combinations with Conventional Chemotherapeutic, Radiotherapeutic, Anti-Angiogenic Agents or Histone Deacetylase Inhibitors (HDACi)

The present invention also provides combined therapies in which a conjugated nucleic acid molecule of the invention is used simultaneously with, before, or after surgery or radiation treatment; or is administered to patients with, before, or after a conventional chemotherapeutic, radiotherapeutic or anti-angiogenic agent, HDAC inhibitor (such as belinostat) or targeted immunotoxin.

The present invention also provides a method of treating cancer by administering to a patient in need thereof a conjugated nucleic acid molecule of the present invention in combination with a conventional chemotherapeutic, radiotherapeutic or anti-angiogenic agent, or HDACi or targeted immunotoxin. The invention also concerns a pharmaceutical composition comprising a conjugated nucleic acid molecule of the invention and a conventional chemotherapeutic, radiotherapeutic or anti-angiogenic agent, or HDACi or targeted immunotoxin, more particularly for use in the treatment of cancer. The invention also concerns a product comprising a conjugated nucleic acid molecule of the invention and a conventional chemotherapeutic, radiotherapeutic or anti-angiogenic agent, or HDACi, or targeted immunotoxin as a combined preparation for simultaneous, separate or sequential use, more particularly for use in the treatment of cancer.

Further aspects and advantages of the present invention will be disclosed in the following experimental section, which should be regarded as illustrative and not limiting the scope of the present application. A number of references are cited in the present specification; each of these cited references is incorporated herein by reference.

### EXAMPLES

### Example 1: Synthesis of Exemplary Nucleic acid Molecules

### Example 1-1: Synthesis of OX401

The synthesis of OX401 was based on standard solid-phase DNA synthesis using solid phosphoramidite chemistry (dA(Bz); dC(Bz); dG(Ibu); dT (−)), HEG and Cho16 phosphoramidites.

Detritylation steps were performed with 3% DCA in toluene, oxidations were performed with 50 mM iodine in pyridine/water 9/1 and sulfurizations were performed with 50 mM DDTT in pyridine/ACN 1/1. The capping was done with 20% NMI in ACN, together with 20% Ac₂O in 2,6-lutidine/ACN (40/60). The cleavage and deprotection are performed with respectively 20% diethylamine in ACN to remove cyanoethyl protecting groups on phosphates/thiophosphates for 25 min and concentrated aqueous ammonia for 18 hours at 45° C.

The crude solution was loaded onto a preparative AEX-HPLC column (TSK gel SuperQ 5PW20). Purification was then performed eluting with a salt gradient of sodium bromide at pH 12 containing 20% acetonitrile by volume. After pooling of the fractions, desalting was performed by TFF on regenerated cellulose.

Purity of OX401: 91.8% by AEX-HPLC; Molecular weight by ESI-MS: 11046.5 Da.

### HEG Phosphoramidite (Hexaethylene Glycol Phosphoramidite)

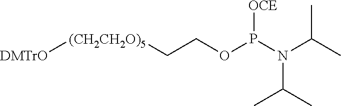

(No CLP-9765, ChemGenes Corp)

### Chol6Phosphoramidite

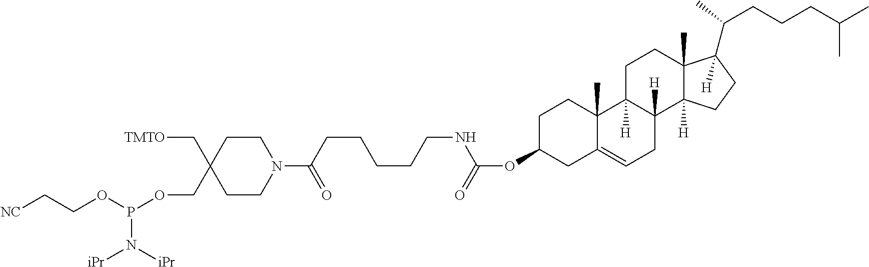

(No 51230, AM Chemicals)

### Example 1-2: Synthesis of OX402

The synthesis, cleavage and deprotection steps are identical to OX401.

The crude solution was loaded onto a preparative AEX-HPLC column. Purification was then performed eluting with a salt gradient of sodium bromide at pH 8 containing 20% acetonitrile by volume. After pooling of the fractions, desalting was performed by SEC on stabilized cellulose.

Purity of OX402: 92.2% by AEX-HPLC; Molecular weight by ESI-MS: 7340.7 Da.

### Example 1-3: Synthesis of Backbone=OX499

The synthesis of OX499 was based on the same protocol as OX401 except for the use of dC(Ac) instead of dC(Bz) and NH₂—C6 phosphoramidite.

The cleavage and deprotection are performed with respectively 20% diethylamine in ACN and AMA (NH₃, methylamine).

The crude solution was first purified on a preparative AEX-HPLC column at pH 12, then by RP-HPLC at pH 7. After pooling of the fractions, desalting was performed by SEC on stabilized cellulose.

Purity of OX499: 95.7% by AEX-HPLC; Molecular weight by ESI-MS: 10637.0 Da.

### Example 1-4: Synthesis of OX403

SV119 (0.123 mmol) has been conjugated first with an activated PEG of 9 units (1.2 eq) before coupling with OX499. The final conjugated compound OX403 has been purified using a RP column

### Example 1-5: Synthesis of OX404

Synthesis was performed following the same synthesis route as for OX403

### Example 1-6: Synthesis of OX406

The synthesis, cleavage, deprotection and purification (AEX-HPLC column) steps are identical to those of OX401.
Purity of OX406: 96.5% by AEX-HPLC; Molecular weight by ESI-MS: 11054.3 Da.

### Example 1-7: Synthesis of OX407

The synthesis, cleavage, deprotection and purification (AEX-HPLC column) steps are identical to those of OX401.
Purity of OX407: 95.7% by AEX-HPLC; Molecular weight by ESI-MS: 10966.2 Da.

### Example 1-8: Synthesis of OX408

The synthesis, cleavage, deprotection and purification (AEX-HPLC column) steps are identical to those of OX401.
Purity of OX408: 88.4% by AEX-HPLC; Molecular weight by ESI-MS: 10982.2 Da.

### Example 1-9: Synthesis of (OX410)

The synthesis, cleavage, deprotection and purification steps are identical to those of OX401.
The crude solution was first purified on a preparative AEX-HPLC column, then by RP-HPLC column.
Purity of OX410: 83.6% by AEX-HPLC; Molecular weight by ESI-MS: 11051.3 Da.

### Example 1-10: Synthesis of (OX411)

The synthesis, cleavage, deprotection and purification steps are identical to those of OX401.
The crude solution was first purified on a preparative AEX-HPLC column, then by RP-HPLC column.
Purity of OX411: 83.1% by AEX-HPLC; Molecular weight by ESI-MS: 11051.3 Da.

### Example 2: OX401 Hyperactivates PARP but not DNA-PK

Materials and Methods

Cell Culture

Triple negative breast cancer cell line MDA-MB-231 was purchased from ATCC and grown according to the supplier's instructions. Briefly, MDA-MB-231 cells were grown in L15 Leibovitz medium supplemented with 10% fetal bovine serum (FBS) and maintained in a humidified atmosphere at 37° C. and 0% CO2.

ELISA Anti-PARylation

A sandwich ELISA was used to detect Poly(ADP-Ribose) (PAR) polymers. Cells were boiled in Tissue Protein Extraction (T-PER) Buffer (Thermo Scientific) supplemented with 1 mM PMSF (Phenylmethanesulfonyl Fluoride, Sigma). Cell extracts were then diluted in Superblock buffer (Thermo Scientific) prior to the ELISA Assay. A 96-well polystyrene plate (Thermo Scientific Pierce White Opaque) was coated with 100 l per well carbonate buffer (1.5 g/l sodium carbonate Na₂CO₃, 3 g/l NaHCO₃) containing the capture antibody (mouse anti-PAR at 4 μg/ml, Trevigen 4335) overnight at 4° C., after which it was washed with PBST solution. The wells were then overcoated with Superblock at 37° C. for 1 h. Then, 10 l of cell extract was added to 65 μL of Superblock and were applied to each well in triplicate and incubated overnight at 4° C., after which it was washed with PBST solution. Then the detection antibody (Rabbit anti-PAR, Trevigen 4336, diluted 1/1000 in PBS/2% milk/1% mouse serum) was added and incubated for 1 h at room temperature. After washing secondary antibody HRP-conjugated anti-rabbit (Abcam, ab97085, diluted 1/5000 in PBS/2% milk/1% mouse serum) was applied to each well for 1 h. To readout, 75 l of substrate for the enzyme (Supersignal Pico, Pierce) was added to each well. The chemiluminescent reading was determined immediately.

ELISA anti-γH2AX

A sandwich ELISA was used to detect the phosphorylated form of histone H2AX (γH2AX). Cells were boiled in Tissue Protein Extraction (T-PER) Buffer (Thermo Scientific) supplemented with 1 mM PMSF (Phenylmethanesulfonyl Fluoride, Sigma). Cell extracts were then diluted in Superblock buffer (Thermo Scientific) prior to the ELISA Assay. A 96-well polystyrene plate (Thermo Scientific Pierce White Opaque) was coated with 100 l per well carbonate buffer (1.5 g/l sodium carbonate Na₂CO₃, 3 g/l NaHCO₃) containing the capture antibody (mouse anti-γH2AX at 4 g/ml, Millipore 05-636) overnight at 4° C., after which it was washed with PBST solution. The wells were then overcoated with Superblock at 37° C. for 1 h. Then, 50 l of cell extract were applied to each well in triplicate and incubated for 2 h at 25° C., after which it was washed with PBST solution. Then the detection antibody (Rabbit anti-H2AX, Abcam ab11175, diluted 1/500 in PBS/2% milk) was added and incubated for 1 h at 25° C. After washing, anti-rabbit secondary antibody HRP-conjugated (Abcam, ab97085, diluted 1/20000 in PBS/2% milk) was applied to each well for 1 h at 25° C. To readout, 75 l of substrate for the enzyme (Supersignal Pico, Pierce) was added to each well. The chemiluminescent reading was determined immediately.

Statistical Analysis

All statistical analyses were performed with a two-tailed Student t test.

Results

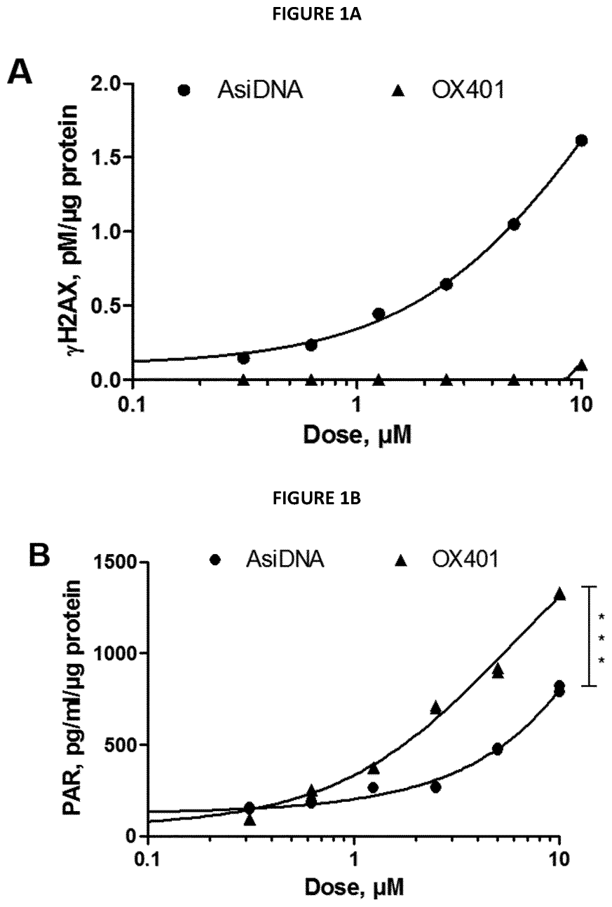

The inventors first analyzed OX401 activity in MDA-MB-231 cells by monitoring the activation of DNA-dependent protein kinase (DNA-PK) and Poly-(ADP-ribose) polymerase (PARP). Both enzymes are activated to modify their targets after interacting with the AsiDNA™ DNA moiety, which mimics a double-strand break. MDA-MB-231 cells treated with AsiDNA showed dose-dependent phosphorylation of the histone H2AX (γH2AX) and Poly(ADP-Ribose) (PAR) polymer accumulation (PARylation) after treatment, caused by DNA-PK and PARP activation, respectively (FIG. 1A, B). Cells treated with OX401 did not interact and activate DNA-PK enzyme, compared to AsiDNA™ (FIG. 1A). However, OX401 highly hyperactivated PARP enzymes and induced a dose-dependent PARylation two fold higher than AsiDNA™ (FIG. 1B). Thus, they observed target engagement in MDA-MB-231 cells shown by false DNA damage signaling (PARylation) induced by OX401.

### Example 3: OX401 Displays a Specific Antitumor Activity

Materials and Methods

Cell Culture

Cell cultures were performed with the triple negative breast cancer cell line MDA-MB-231, the histiocytic lymphoma cell line U937 and the non-tumor mammary cell line MCF-10A. Cells were grown according to the supplier's instructions. Cell lines were maintained at 37° C. in a humidified atmosphere at 5% CO₂, except the MDA-MB-231 cell line which was maintained at 0% CO₂.

Drug Treatment and Measurement of Cellular Survival

MDA-MB-231 (5.10³ cells/well), MCF-10A (5.10³ cells/well) and U937 (2.10⁴ cells/well) were seeded in 96 well-plates and incubated 24 hours at +37° C. before drug addition with increasing concentrations for 4 to 7 days. Following drug exposure, cell survival was measured using the XTT assay (Sigma Aldrich). Briefly, the XTT solution was added directly to each well containing cell culture and the cells incubated for 5 hours at 37° C. before reading the absorbance at 490 nm and 690 nm using a microplate reader (BMG Fluostar, Galaxy). Cell survival was calculated as the ratio of living treated cells to living mock-treated cells. The IC₅₀ (which represents the dose at which 50% of the cells are viable) was calculated by a non-linear regression model using GraphPad Prism software (version 5.04) by plotting the percentage viability against the Log of the drug concentration on each cell line.

Results

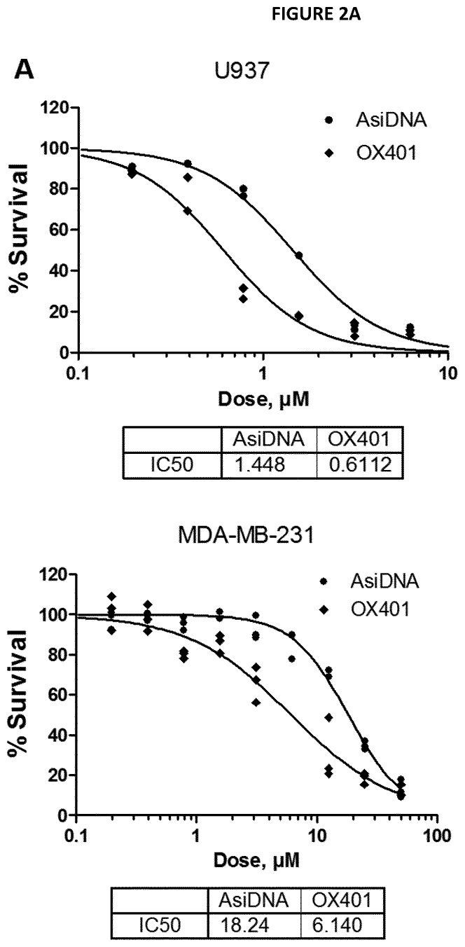

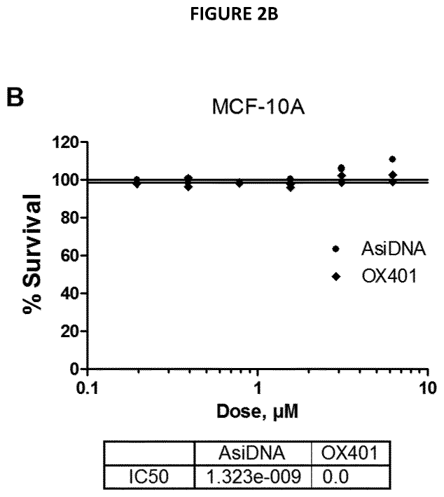

As OX401 induces only PARP target engagement and not DNA-PK compared to AsiDNA™, we wanted to ensure that it displays an interesting antitumor activity. Tumor (MDA-MB-231, U937) and non-tumor (MCF-10A) cells were treated with AsiDNA (Black) or OX401 (dark grey) and survival was measured 4 days (U937) or 7 days (MDA-MB-231 and MCF-10A) after treatment using the XTT assay (FIG. 2). OX401 displayed higher antitumor activity than AsiDNA™, as shown by OX401 IC₅₀ values 3-fold lower than AsiDNA™ (FIG. 2A). The MCF10A non-tumor cells were insensitive to OX401, highlighting its tumor specificity (FIG. 2B). Absence of any effect in non-tumor cells predicts a non-toxicity and a high safety of OX401 treatment in normal tissues.

### Example 4: OX401 Induces a Tumor Immune Response

Materials and Methods

Cell Culture

Cell cultures were performed with the triple negative breast cancer cell line MDA-MB-231 and the non-tumor mammary cell line MCF-10A. Cells were grown according to the supplier's instructions. Cell lines were maintained at 37° C. in a humidified atmosphere at 5% CO₂, except the MDA-MB-231 cell line which was maintained at 0% CO₂.

Long Term Treatment with OX401 or AsiDNA™

Cells were seeded in 6-well culture plates at appropriate densities and incubated 24 h at 37° C. before OX401 or AsiDNA™ addition at a concentration of 5 μM. Cells were harvested on day 7 after treatment, washed to remove the drug, and again seeded in 6-well culture plates for recovery during 7 days. A period of one week treatment/one week release consists in a one treatment cycle. After each treatment cycle, further analyses were performed (micronuclei quantification; western blot; ELISA; flow cytometry).

Western Blot Analysis

Cells treated for one cycle with OX401 or AsiDNA™ (5 μM) were harvested, seeded at appropriate densities and then re-treated for 48 hours. Cells were then lysed in RIPA buffer (150 mM NaCl, 50 mM Tris-base, 5 mM EDTA, 1% NP-40, 0.25% deoxycholate, pH 7.4) with protease and phosphatase inhibitors (Roche Applied Science, Germany). Protein concentrations were measured using the BCA protein assay (Thermo Fisher Scientific, USA). Equal amounts (15 μg) of the protein were electrophoresed using SDS-PAGE (12% gel), transferred to nitrocellulose membranes, blocked with 5% skim milk in TBS Tween 1% for 1 hour at room temperature and then incubated with primary antibodies overnight at 4° C. Following washes with TBS/Tween 1%, membranes were incubated with the secondary antibody for 1 hour at room temperature. The bound antibodies were detected using the Enhanced Chemiluminescence western blotting substrate kit (Ozyme, USA). Western blotting was done with the following antibodies: primary monoclonal rabbit anti-sting (dilution 1/1,000; CST-13647), primary monoclonal mouse anti-PD-L1 (dilution 1/1,000; abeam ab238697), primary monoclonal mouse anti-pactin (dilution 1/10,000, Sigma A1978), secondary goat anti-rabbit IgG, HRP conjugate (dilution 1/2,000, Millipore 12-348) and secondary goat anti-mouse IgG, HRP conjugate (dilution 1/2,000, Millipore 12-349).

Flow Cytometry to Detect Cell Surface PD-L1

Cells treated for one cycle with OX401 or AsiDNA™ (5 μM) were harvested, seeded in 6-well plates at appropriate densities and then re-treated for 48 hours. Cells were then washed with PBS and incubated for one hour at 4° C. with anti-PD-L1 monoclonal antibody Alexa Fluor 488-conjugated (CST—14772). Cells are then washed with PBS and fluorescence intensities were determined with a Guava easyCyte (Merck). Data were analyzed using FlowJo software (Tree Star, Calif.).

Micronuclei Quantification

Micronuclei result from chromosomal breakage or spindle damage. They arise in the nuclei of daughter cells following cell division and form single or multiple micronuclei in the cytoplasm. Cells treated for one cycle with OX401 or AsiDNA™ (5 μM) were grown on cover slips in a Petri dish. Cells were then fixed with PFA (4%), permeabilized with Triton (0.5%), and stained with DAPI (0.5 mg/mL). The frequency of micronuclei was estimated as the percentage of cells with micronuclei over the total number of cells. At least 1,000 cells were analyzed for each condition.

ELISA to Detect CCL5 Chemokine

Cells treated for one cycle with OX401 or AsiDNA™ (5 μM) were harvested, seeded in 6-well plates at appropriate densities and then re-treated for 48 hours. Cell culture supernatants were then centrifuged at 2,000×g for 10 minutes to remove debris. The 96 well plate strips included with the kit (Human SimpleStep ELISA Kit—Abcam—ab174446) are supplied ready to use. 50 μl of each supernatant were added to each well in duplicate with 50 μl of the Antibody cocktail and then, incubated for 1 h at RT on a plate shaker set to 400 rpm, after which it was washed with 1× Wash buffer PT. Then 100 μl of TMB substrate were added to each well and incubated for 10 min in the dark on a plate shaker set to 400 rpm. 100 μl of stop solution were then added to each well for 1 minute on a plate shaker and the optical absorbance was determined at 450 nm.

ELISA to Detect CXCL10 (IP-10) Chemokine

Cells treated for one cycle with OX401 or AsiDNA™ (5 μM) were harvested, seeded in 6-well plates at appropriate densities and then re-treated for 48 hours. Cell culture supernatants were then centrifuged at 1,000×g for 10 minutes to remove debris. The 96 well plate strips included with the kit (IP-10 (CXCL10) Human ELISA Kit—Abcam—ab83700) are supplied ready to use. 100 μl of each supernatant were added to each well in duplicate and then, incubated for 2 h at RT, after which it was washed with 1× Wash buffer PT. Then 50 μl of Biotinylated ant-IP-10 were added to each well and incubated for 1 h, after which it was washed with 1× Wash buffer PT. Then 100 μl of 1× Streptavidin-HRP solution were added to each well and incubated for 30 min and washed with 1× Wash buffer PT. Then 100 μl of Chromogen TMB substrate solution were added to each well and incubated 10-20 minutes in the dark. 100 μl of Stop Reagent were added to each well and the optical absorbance was determined immediately at 450 nm.

Statistical Analysis

All statistical analyses were performed with a two-tailed Student t test.

Results

As OX401 is a double-stranded DNA, we wondered if it could be recognized by innate immunity pathways. Stimulator of interferon genes (STING) is a cytosolic receptor that senses both exogenous and endogenous cytosolic DNA and triggers type I interferon and pro inflammatory cytokine responses. Therefore, the inventors evaluated the activation of STING pathway in cells treated with OX401. Intriguingly, OX401 is not recognized as an exogenous DNA by the STING pathway, and did not trigger direct induction of chemokines nor interferon cytokines (data not shown).

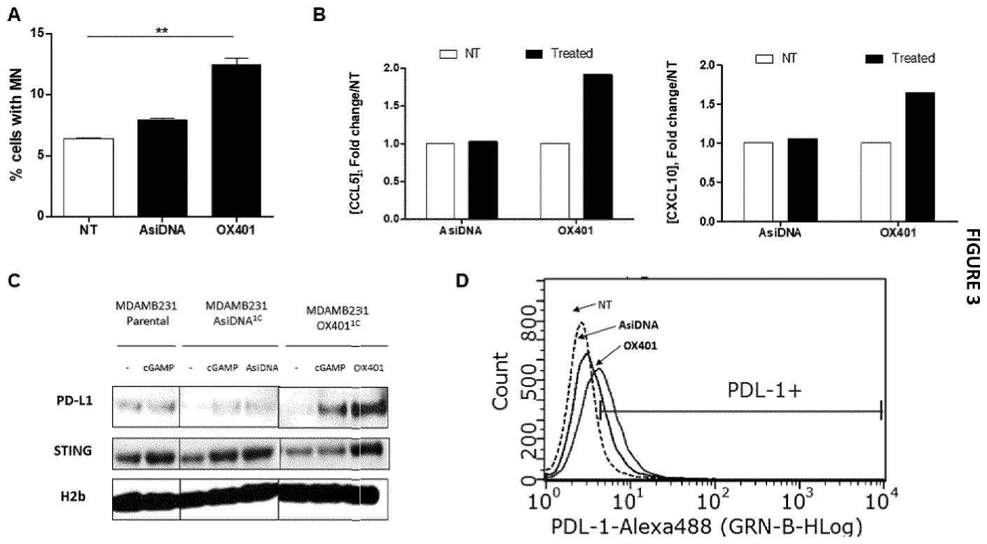

Since short term treatment by OX401 didn't induce directly an antitumor immune response, the inventors hypothesized that long term treatment could trigger indirectly a STING-dependent immune response through the accumulation of unrepaired DNA structure. In agreement with this hypothesis, cells treated for a long term (one cycle of one week treatment/one week release) with OX401 showed a two-fold significant increase of % of cells with micronuclei compared to non-treated or AsiDNA™-treated cells (FIG. 3A). To validate the link between micronuclei increase and STING pathway activation, the inventors analyzed the release of CCL5 and CXCL10 target chemokines. Interestingly, long term treated cells with OX401 secreted two-fold more CCL5 than non-treated cells, and 1.5-fold more CXCL10 (FIG. 3B). AsiDNA-treated cells did not show more secretion of CCL5 or CXCL10 (FIG. 3B). Among the consequences of STING pathway activation in tumor cells is PD-L1 (programmed death ligand 1) up-regulation, probably a reaction to protect against the immune system. The inventors analyzed the level of total PD-L1 or cell-surface associated PD-L1 in long term treated cells. OX401-treated cells showed a high increase in total level (FIG. 3C) and membrane associated PD-L1 (FIG. 3D) compared to parental cells of AsiDNA™-treated cells.

Taken together, these results demonstrate that OX401 triggers an indirect STING-pathway activation through micronuclei induced accumulation, and pave the way for combined treatments with anti-PD-L1 therapies.

### Example 6: Pharmaceutical Properties/PK/PD Experiments

Injection of OX401 at a dose of 2 mg by intravenous (iv) route in mice leads to a plasmatic concentration maximum (CMAX) of 8 μM as measured by HPLC method. Unexpectedly this Cmax is 40 time higher than the Cmax obtained in the same experimental conditions with AsiDNA.

### Example 7: OX402 Hyperactivates PARP—Smaller but as Active as OX401

Materials and Methods

Cell Culture

Triple negative breast cancer cell line MDA-MB-231 was purchased from ATCC and grown according to the supplier's instructions. Briefly, MDA-MB-231 cells are grown in L15 Leibovitz medium supplemented with 10% fetal bovine serum (FBS) and maintained in a humidified atmosphere at 37° C. and 0% CO₂.

### ELISA Anti-PARylation

A sandwich ELISA was used to detect Poly(ADP-Ribose) (PAR) polymers. Cells were boiled in Tissue Protein Extraction (T-PER) Buffer (Thermo Scientific) supplemented with 1 mM PMSF (Phenylmethanesulfonyl Fluoride, Sigma). Cell extracts were then diluted in Superblock buffer (Thermo Scientific) prior to the ELISA Assay. A 96-well polystyrene plate (Thermo Scientific Pierce White Opaque) was coated with 100 l per well carbonate buffer (1.5 g/l sodium carbonate Na₂CO₃, 3 g/l NaHCO₃) containing the capture antibody (mouse anti-PAR at 4 μg/ml, Trevigen 4335) overnight at 4° C., after which it was washed with PBST solution. The wells were then overcoated with Superblock at 37° C. for 1 h. Then, 10 l of cell extract was added to 65 μL of Superblock and were applied to each well in triplicate and incubated overnight at 4° C., after which it was washed with PBST solution. Then the detection antibody (Rabbit anti-PAR, Trevigen 4336, diluted 1/1000 in PBS/2% milk/1% mouse serum) was added and incubated for 1 h at room temperature. After washing secondary antibody HRP-conjugated anti-rabbit (Abcam, ab97085, diluted 1/5000 in PBS/2% milk/1% mouse serum) was applied to each well for 1 h. To readout, 75 μl of substrate for the enzyme (Supersignal Pico, Pierce) was added to each well. The chemiluminescent reading was determined immediately.

Results

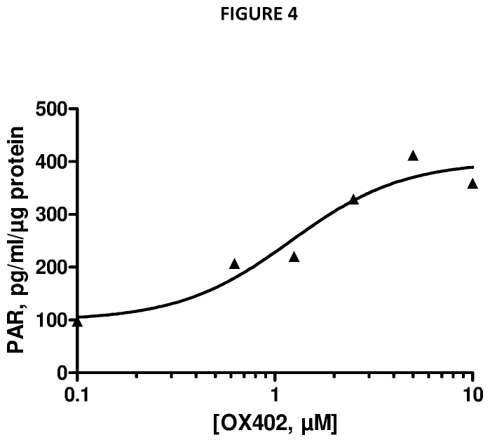

The inventors also analyzed the minimal sequence length required to activate PARP and induce the false damage signaling (PARylation). MDA-MB-231 cells were treated during 24 h with OX402, a 10 bases pair (bp) molecule, and PARP activation was monitored using an anti-PARylation ELISA assay. MDA-MB-231 cells treated with OX402 showed a dose-dependent PARylation caused by PARP engagement and activation (FIG. 4). Thus, 10 bp molecules are sufficient to hijack and activate PARP.

### Example 8: OX401 Induces Intracellular NAD⁺ Depletion

PARP proteins bind to DSBs with a high affinity. Upon binding, PARP are auto “PARylated” and activate other target proteins by the addition of polymers of Poly(ADP-Ribose) (PAR) referred to as PARylation. The kinetics of PARP activation in MDA-MB-231 and MRC5 cells treated with OX401 were studied by monitoring proteins PARylation.

Materials and Methods

Cell Culture

Cell cultures were performed using the triple negative breast cancer cell line MDA-MB-231, and the non-tumor MRC5 primary lung fibroblasts. All cell lines were purchased from ATCC and grown according to the supplier's instructions in a humidified atmosphere at 37° C. and 5% CO2, except for MDA-MB-231 (37° C. and 0% CO2).

Cell Treatment with OX401 and Assessment of Survival

MDA-MB-231 or MRC5 cells were seeded in 60 mm diameter culture plates at appropriate densities and incubated over-night at 37° C. Cells were then treated with 5 μM of OX401 during 48 hours, 7 days and 13 days before washing, harvesting and counting using trypan blue (4%) cell staining assay and Eve automatic cell counter (VWR) for further analysis.

Measurement of Intracellular Levels of NAD⁺

NAD content was determined using the NAD/NADH-Glo Assay kit (Promega, G9071) according to the manufacturer's instructions. The principle of the assay consists on a succession of transformations: first, the NAD cycling enzyme modifies NAD⁺ to NADH which is used by the reductase to convert a substrate into luciferin. Then, the luciferase uses the Luciferin to produces light. So the luminescence produced is proportional to the amount of NAD⁺ present in the cell.

Briefly, MDA-MB-231 or MRC5 cells treated with OX401 (5 μM) during 48 hours, 7 days or 13 days were harvested and seeded in 96-well plates (5.10⁴ cells/well). Cells were then lysed using a 1% DTAB buffer and 25 μL of HCl 0,4M was added in each well and incubated for 15 min at 60° c. and 10 min at room temperature. 25 μL of the detection reagent Trizma and 100 μL of the NAD detection reagent was added in each well. The resulting luminescent signals were measured on a microplate reader (Enspire™ Perkin-Almer).

Western Blot Analysis

MDA-MB-231 or MRC5 cells treated with OX401 (5 μM) during 48 hours, 7 days or 13 days were harvested and lysed in RIPA buffer (150 mM NaCl, 50 mM Tris-base, 5 mM EDTA, 1% NP-40, 0.25% deoxycholate, pH 7.4) with protease and phosphatase inhibitors (Roche Applied Science, Germany). Protein concentrations were measured using the BCA protein assay (Thermo Fisher Scientific, USA). Equal amounts (15 μg) of the protein were electrophoresed using SDS-PAGE (12% gel), transferred to nitrocellulose membranes, blocked with 5% skim milk in TBS Tween 1% for 1 hour at room temperature and then incubated with primary antibodies overnight at 4° C. Following washes with TBS/Tween 1%, membranes were incubated with the secondary antibody for 1 hour at room temperature. The bound antibodies were detected using the Enhanced Chemiluminescence western blotting substrate kit (Ozyme, USA). Western blotting was done with the following antibodies: anti-Pan-ADP Ribose binding reagent (dilution 1/1,500; Millipore MABE1016), primary monoclonal mouse anti-pactin (dilution 1/10,000, Sigma A1978), secondary goat anti-rabbit IgG, HRP conjugate (dilution 1/2,000, Millipore 12-348) and secondary goat anti-mouse IgG, HRP conjugate (dilution 1/2,000, Millipore 12-348).

Results

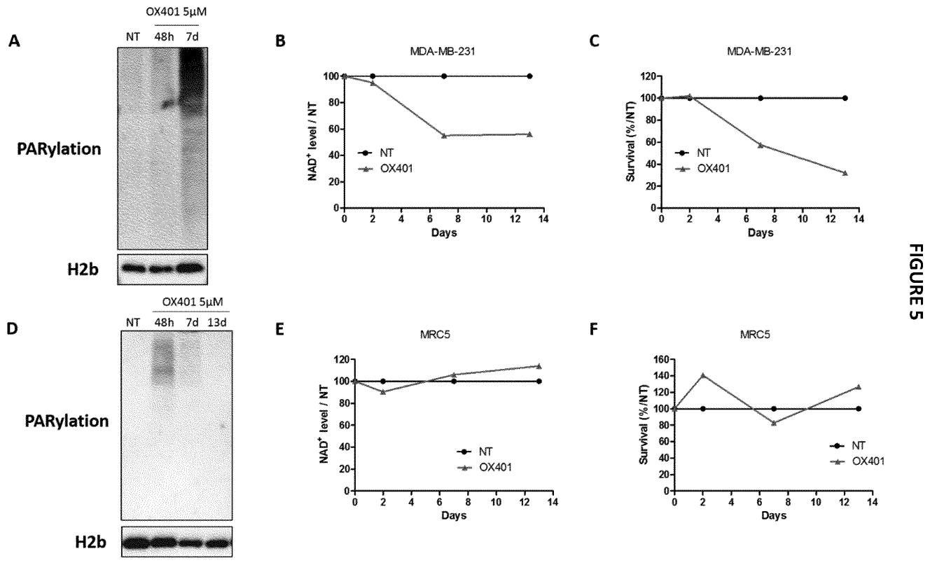

MDA-MB-231 cells treated with OX401 (5 μM) showed an accumulation of PARylated proteins after treatment, with a pic 7 days after treatment (FIG. 5A). As Nicotinamide adenine dinucleotide (NAD⁺) is used as a substrate by PARP for PARylation of its target proteins, intracellular NAD⁺ levels were analyzed after OX401 treatment. OX401 induced a high NAD⁺ consumption in MDA-MB-231 cells with a maximum of 55% NAD⁺ level compared to non-treated cells 7 days after treatment, which was maintained until 13 days after treatment (FIG. 5B). Giving this profound OX401-induced NAD⁺ deficiency, we hypothesized that tumor cells fail to regulate and maintain the homeostasis of NAD⁺ levels under OX401 treatment, leading to cell death. To test this hypothesis, we analyzed MDA-MB-231 cell survival under OX401 treatment. No effect on cell survival was observed 48 hours after OX401 treatment, which is in accordance with the very low decrease in NAD⁺ level at this time. A striking effect on cell survival was observed 7 and 13 days after treatment (57% and 32% survival compared to non-treated cells, respectively), validating the importance of NAD⁺ levels for cell survival (FIG. 5C). All these effects were specific to tumor cells, since no NAD⁺ depletion nor cell death was observed in OX401-treated MRC5 non-tumor cells (FIG. 5D-F).

Taken together, these results indicate that long term treatment with OX401 induces both PARP hyperactivation and NAD⁺ consumption. An acute OX401-induced drop in NAD⁺ level below a threshold compatible with cell survival would outstrip the cellular NAD⁺ replenishment capacities and trigger a massive tumor cell death.

### Example 10

### OX401 Disturbs the Homologous Recombination (HR) Repair Pathway

As OX401 lures PARP and induces a false DNA damage PARylation signaling, inventors tested if OX401 could trigger a rapid accumulation of DNA damages.

Homologous recombination (HR) repair pathway is an error-free repair pathway essential to maintain genetic stability and intact DNA information. HR is a well-organized multi-step machinery that consume a large amount of cellular energy. As OX401 triggers a high NAD⁺ consumption and therefore induces a metabolic disequilibrium in tumor cells (Example 9), inventors hypothesized that it could disturb the HR repair machinery very dependent of energy. To test this hypothesis, the HR repair efficiency (by the detection of Rad51 protein recruitment to sites of DSBs) was analyzed after OX401 treatment.

Materials and Methods

Cell Culture

Cell cultures were performed with the triple negative breast cancer cell line MDA-MB-231. Cells were grown in complete L15 Leibovitz medium and maintained at 37° C. in a humidified atmosphere at 0% Co₂.

Homologous Recombination Pathway Activity Analysis

For immunostaining, cells are seeded on cover slips (Menzel, Braunschweig, Germany) at a concentration of 5×10⁵ cells and incubated at 37° C. during 1 day. Cells are then treated with olaparib (5 μM)+/−OX401 (5 μM). 48 h after treatment, cells are fixed for 20 min in 4% paraformaldehyde/Phosphate-Buffered Saline (PBS 1×), permeabilized in 0.5% Triton X-100 for 10 min, blocked with 2% bovine serum albumin/PBS 1× and incubated with primary antibody for 1 h at 4° C. All secondary antibodies were used at a dilution of 1/200 for 45 min at Room Temperature (RT), and DNA was stained with 4′, 6-diamidino-2-phenylindole (DAPI). The following antibodies were used: primary monoclonal mouse anti-phospho-H2AX (Millipore, Guyancourt, France), anti-Rad51 rabbit antibody (Merk Millipore, Darmstadt, Allemagne), secondary goat anti-mouse IgG conjugated with Alexa-633 (Molecular Probes, Eugene, Oreg., USA) and secondary goat anti-rabbit IgG conjugated with Alexa-488 (Molecular Probes, Eugene, Oreg., USA).

Analysis of Drug-Induced DNA Damage by Flow Cytometry

Cells were treated with OX401 (5 μM) or Olaparib (5 μM) for 48 hours and then fixed and permeabilized with cold (−20° C.) 70% ethanol for at least 2 hours. After washing with PBS, the cells were further permeabilized with 0.5% Triton in PBS for 20 minutes at RT, washed in PBS, and incubated with anti-γ-H2AX antibody (05-636 Millipore) in 2% BSA in PBS. After washing with PBS, and cells were incubated with an Alexa Fluor 488-conjugated secondary antibody. Fluorescence intensities were determined with a Guava EasyCyte cytometer (Luminex). Data were analyzed using FlowJo software (Tree Star, Calif.).

Results

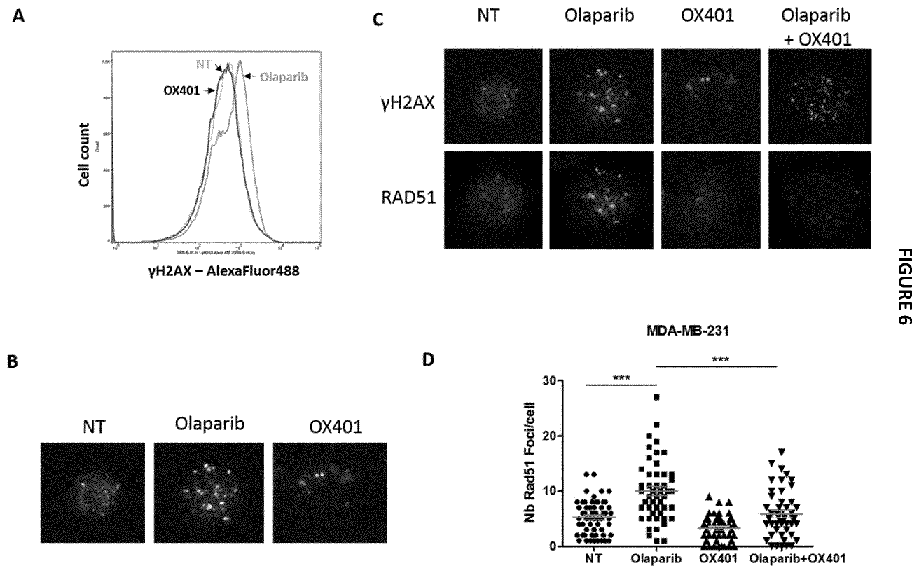

As expected, olaparib induced an accumulation of double-strand breaks (DSBs) 48 h after treatment in MDA-MB-231 cells, as showed by the high phosphorylation of histone H2AX (γH2AX) measured by flow cytometry (FIG. 6A) or by the detection of TH2AX Foci by immunofluorescence (FIG. 6B). In comparison, OX401 did not induce an increase of γH2AX DSB biomarker and therefore did not trigger a direct DSBs accumulation (FIG. 6A, B).

MDA-MB-231 cells treated with olaparib (5 μM) for 48 h showed an accumulation of γH2AX Foci that co-localize with Rad51 foci, indicating a repair of olaparib-induced DSBs by the HR repair pathway (FIG. 6C). The addition of OX401 (5 μM) significantly reduced the formation of Rad51 foci induced by olaparib (FIG. 6C, D), demonstrating that OX401 effectively disturbs the HR pathway probably through energy depletion consecutive to metabolism disequilibrium.

### Example 11

Tumor Cells do not Acquire a Resistance Against OX401

It is currently accepted that cancer is subject to the evolutionary processes laid out by Charles Darwin in his concept of natural selection. Natural selection is the process by which nature selects certain physical attributes, or phenotypes, to pass on to offspring to better “fit” the organism to the environment. Under the selective pressure of targeted therapies, resistant populations of cancer cells invariably evolve giving rise to “resistant clones” that have adapted to the new environment induced by the treatment. It's also well established that tumor cells need a lot of energy to develop resistance to anti-cancer treatments. Since OX401 induces NAD+ depletion and metabolic disequilibrium (Example 8), inventors tested whether or not cells develop resistance to OX401.

Materials and Methods

Cell Culture

Cell cultures were performed with the lymphoma cell line U937. Cells were grown in complete RPMI medium supplemented with 10% FBS and 1% Penicillin/Streptomycin and maintained at 37° C. in a humidified atmosphere at 5% CO₂. This cell line was chosen according to its high sensitivity to both OX401 and talazoparib.

Selection of Acquired Resistance

For repeated cycles of the treatment to select resistance, U937 cells were seeded at appropriate densities (2.10⁵ cells/mL) and incubated 24 h at 37° C. before addition of the drug at doses corresponding to 10-20% survival compared to non-treated cells. Resistances were selected under 2 μM talazoparib or 1.5 μM OX401. Cells were harvested on day 4 after treatment, washed, and counted after staining with 0.4% trypan blue (Eve™ counting slides, NanoEnTek). After counting, cells were seeded in appropriate culture plates, and allowed to recover (drug free period) for 3 to 7 days. Another cycle of treatment/recovery was then started for up to 4 cycles.

Acquired Resistance Irreversibility—Measurement of Cellular Survival

To assess the acquired resistances irreversibility, U937 parental or resistant cells were seeded in 96 well-plates (2.10⁴ cells/well) and treated with increasing concentrations of talazoparib for 4 days. Following drug exposure, cell survival was measured using the XTT assay (Sigma Aldrich). Briefly, the XTT solution was added directly to each well containing cell culture and the cells incubated for 5 hours at 37° C. before reading the absorbance at 490 nm and 690 nm using a microplate reader (BMG Fluostar, Galaxy). Cell survival was calculated as the ratio of living treated cells to living mock-treated cells. The IC50 (which represents the dose at which 50% of the cells are viable) was calculated by a non-linear regression model using GraphPad Prism software (version 5.04) by plotting the percentage viability against the Log of the drug concentration on each cell line.

Results

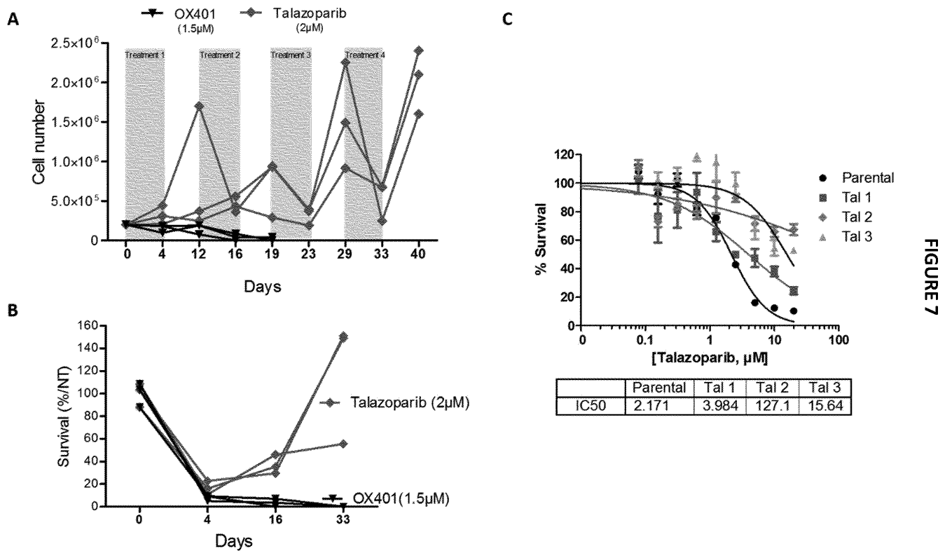

Cycles of treatment with OX401 or talazoparib were performed on U937 cells. Cells treated with talazoparib recovered during amplification periods, whereas cells treated with OX401 didn't grow during drug-free amplification periods (FIG. 7A). Cells treated with talazoparib developed an acquired resistance during cycles of treatment, with a cell survival evolving from 10% after the first cycle to more than 50% survival after the forth cycle of treatment (33 days after treatment start) (p<0.01) (FIG. 7B). To assess the irreversible resistance status to talazoparib, resistant cells was submitted to increasing doses of talazoparib to analyze their sensitivity compared to parental cells. Parental cells, sensitive to talazoparib, showed a low IC50 of 2 μM. Ta11, Ta12 and Ta13 resistant populations showed a higher IC50 of more than 4 μM (FIG. 7C).

### Example 12

### OX401 Amplifies the Anti-Tumor Immune Response

In previous experiments (FIG. 3) inventors showed that long term treatment with OX401 displayed a micronuclei-induced STING pathway activation with an increase of CCL5 and CXCL10 chemokines secretion. To test the effects of these anti-tumor immune effects, co-cultures of tumor cells with freshly isolated T cells were performed and the T cell-induced cytotoxic effects were assessed.

Materials and Methods

Cell Culture

Cell cultures were performed with the triple negative breast cancer cell line MDA-MB-231 and the cervical tumor cell line HeLa. Cells were purchased from ATCC and grown according to the supplier's instructions. Cells were maintained at 37° C. in a humidified atmosphere at 5% CO₂.

Isolation of PBMC

Buffy coats of healthy donors were purchased from the EFS blood center (Paris, France). PBMCs were isolated using the EasySep Direct Human PBMC Isolation kit (19654, Stemcell, France) according to the manufacturer's protocol. The isolated PBMCs were adjusted to a concentration of 5×10⁷ cells/ml in freezing medium (10% DMSO and 90% FBS), from which 1 ml aliquots were dispensed into cryogenic vials and stored in liquid nitrogen at −196° C. until needed.

Isolation of T Lymphocytes from PBMC

T lymphocytes were isolated from PBMCs using the EasySep Human T cell Isolation Kit (17951, Stemcell, France) according to the manufacturer's protocol. Isolated T cells were suspended in ImmunoCult-XF T cell expansion medium (10981, Stemcell, France) at a concentration of 10⁶ cells/ml and activated using the ImmunoCult Human CD3/CD28/CD2 T cell activator (10970, Stemcell, France) during 24 hours before further experiments.

Co-Cultures of Tumor and T Lymphocytes

MDA-MB-231 cells were seeded in 12-wells cell culture plates (5×10⁴ cells/well) or 60 mm diameter cell culture plates (10⁶ cells/plate) and incubated at 37° C. during 24 hours. Activated T cells were added to tumor cells at an effector to target ratio of 4:1, with or without OX401 (5 μM). Co-cultures were incubated for 48 hours at 37° C. At the end of incubation, each cell type (adherent tumor cells or suspension T cells) was counted and supernatant harvested for cytokine release analysis.

Western Blot Analysis

Cells treated with OX401 (5 μM) with or without T lymphocytes were harvested and then lysed in RIPA buffer (150 mM NaCl, 50 mM Tris-base, 5 mM EDTA, 1% NP-40, 0.25% deoxycholate, pH 7.4) with protease and phosphatase inhibitors (Roche Applied Science, Germany). Protein concentrations were measured using the BCA protein assay (Thermo Fisher Scientific, USA). Equal amounts (15 μg) of the protein were electrophoresed using SDS-PAGE (12% gel), transferred to nitrocellulose membranes, blocked with 5% skim milk in TBS Tween 1% for 1 hour at room temperature and then incubated with primary antibodies overnight at 4° C. Following washes with TBS/Tween 1%, membranes were incubated with the secondary antibody for 1 hour at room temperature. The bound antibodies were detected using the Enhanced Chemiluminescence western blotting substrate kit (Ozyme, USA). Western blotting was done with the following antibodies: primary monoclonal rabbit anti-sting (dilution 1/1,000; CST-13647), primary monoclonal mouse anti-PD-L1 (dilution 1/1,000; abcam ab238697), primary monoclonal mouse anti-pactin (dilution 1/10,000, Sigma A1978), secondary goat anti-rabbit IgG, HRP conjugate (dilution 1/2,000, Millipore 12-348) and secondary goat anti-mouse IgG, HRP conjugate (dilution 1/2,000, Millipore 12-349).

ELISA to Detect CCL5 Chemokine

Cells were treated with OX401 (5 μM) with or without T lymphocytes for 48 hours. Cell culture supernatants were then centrifuged at 2,000×g for 10 minutes to remove debris. The 96 well plate strips included with the kit (Human SimpleStep ELISA Kit—Abcam—ab174446) are supplied ready to use. 50 μl of each supernatant were added to each well in duplicate with 50 μl of the Antibody cocktail and then, incubated for 1 h at RT on a plate shaker set to 400 rpm, after which it was washed with 1× Wash buffer PT. Then 100 μl of TMB substrate were added to each well and incubated for 10 min in the dark on a plate shaker set to 400 rpm. 100 μl of stop solution were then added to each well for 1 minute on a plate shaker and the optical absorbance was determined at 450 nm.

ELISA to Detect Granzyme B Enzyme

Cells were treated with OX401 (5 μM) with or without T lymphocytes for 48 hours. Cell culture supernatants were then centrifuged at 2,000×g for 10 minutes to remove debris. The 96 well plate strips included with the kit (Human SimpleStep Granzyme B ELISA Kit—Abcam—ab235635) are supplied ready to use. 50 μl of each supernatant were added to each well in duplicate with 50 μl of the Antibody cocktail and then, incubated for 1 h at RT on a plate shaker set to 400 rpm, after which it was washed with 1× Wash buffer PT. Then 100 μl of TMB substrate were added to each well and incubated for 10 min in the dark on a plate shaker set to 400 rpm. 100 μl of stop solution were then added to each well for 1 minute on a plate shaker and the optical absorbance was determined at 450 nm.

Results

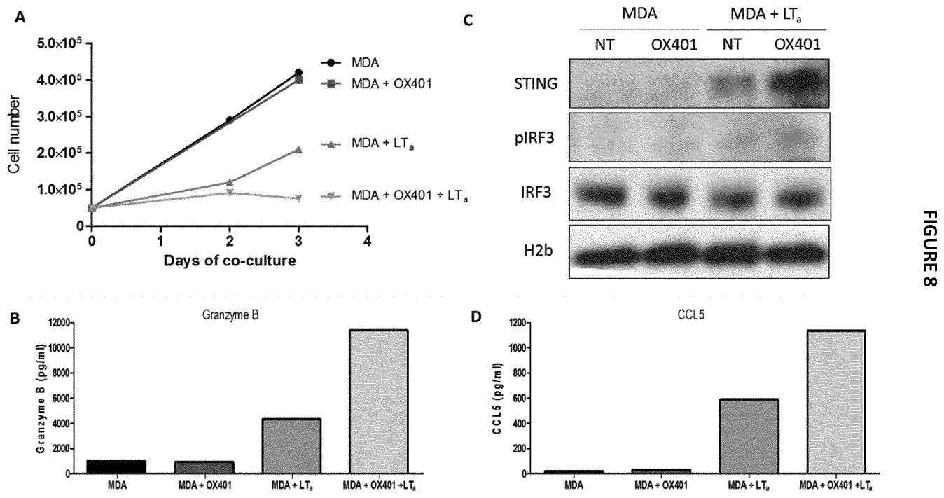

Freshly activated T cells triggered anti-tumor cytotoxic effects 48 and 72 hours after co-culture starting, as revealed by a decrease in MDA-MB-231 tumor cell survival (50% survival compared to MDA-MB-231 cells without T cells) (FIG. 8A). Addition of OX401 to co-cultures further increased T cells-induced anti-tumor cytotoxicity (20% survival compared to non-OX401 treated MDA-MB-231 cells without T cells) (FIG. 8A). Interestingly, cytotoxic T cells secreted higher amounts of Granzyme B in presence of MDA-MB-231 tumor cells treated with OX401 (FIG. 8B), in accordance with the higher cytotoxic efficacy (FIG. 8A). Given the importance of STING pathway activation to trigger a higher immune cells recruitment and anti-tumor cytotoxicity, we analyzed this pathway in tumor/immune cells co-cultures in presence or absence of OX401. After 48 hours of co-cultures, we observed a higher increase of the level of STING proteins in tumor cells treated with OX401 (FIG. 8C). This was associated with a higher IRF3 protein phosphorylation (FIG. 8C) and an increase of secreted CCL5 chemokine (FIG. 8D), indicating a sustained STING pathway activation.

Taken together, these findings demonstrate a high potentiation of anti-tumor cytotoxic T cells by OX401, through a higher STING pathway activation, probably stimulating a better T cell recruitment to the vicinity of tumor cells.

### Example 13: Kinetics of Association (kₒₙ) and Strength of Interaction (K_{D})

Materials and Methods

The interaction of different molecules according the invention with the human poly-[ADP-ribose polymerase 1 protein (PARP-1) (115 kDa) has been characterized by SPR technique using a Biacore T100 instrument from GE Healthcare Life Sciences. The PARP1-His has been captured on Anti-His antibodies immobilized on the surface of the carboxymethylated chip.

Results

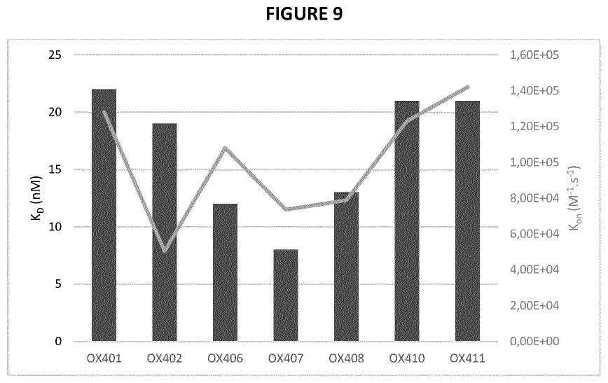

The kinetics of association (kₒₙ) as well as the strength of interaction (K_{D}) are reported in FIG. 9.

OX401, OX410 and OX411 having a modified phosphodiester backbone such as a phosphorothioate linkage (OX401) or both phosphorothioate linkage and FANA modifications (OX410, OX411) on the three first nucleotides on the 3′ and/or 5′ strands, have similar affinities (K_{D}) and kinetics of association (kₒₙ) with PARP-1. OX402 having a phosphorothioate linkage on the three first nucleotides on the 3′ and/or 5′ strands, has similar affinity of association with PARP-1 than above mentioned molecules, but has a lower kinetic of association with PARP-1.

The strength of association seems to be higher with OX406 carrying two FANA modifications in the 3′-strand.

The reduction of number of phosphorothioate modifications to a single nucleotide (OX407 and OX408) increases significantly the strength of interaction (decrease of K_{D} value) and the kinetics of associations (kₒₙ).

From this set of experiments, it was clear that the chemical modifications of the three first nucleotides on the 3′ and/or 5′ strand have a strong influence on the interaction of conjugated nucleic acid molecules with PARP by modulating the strength of interaction (K_{D}) and the kinetics of association (kₒₙ) confirming their strong interest as potential candidates as DNA repair pathway inhibitors, and therefore in the cancer therapy.

1-25. (canceled)

26. A conjugated nucleic acid molecule comprising a double-stranded nucleic acid moiety, the 5′ end of the first strand and the 3′ end of the complementary strand being linked together by a loop, and optionally a molecule facilitating the endocytosis which is linked to the loop,
wherein
the length of the double-stranded nucleic acid moiety is from 10 to 20 base pairs;
the sequence of the double-stranded nucleic acid moiety has less than 80% sequence identity to any gene in a human genome;
the double-stranded nucleic acid moiety comprises deoxyribonucleotides and up to 30% of ribonucleotides or modified deoxyribonucleotides with respect to the total number of nucleotides of the nucleic acid molecule; and
the loop has a structure selected from one of the following formulae:
—O—P(X)OH—O—{[(CH₂)₂—O]_{g}—P(X)OH—O}ᵣ—K—O—P(X)OH—O—{[(CH₂)₂—O]ₕ—P(X)OH—O—}ₛ (I)
with r and s being independently an integer 0 or 1; g and h being independently an integer from 1 to 7 and the sum g+h being from 4 to 7;
with K being 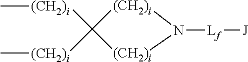
with i, j, k and 1 being independently an integer from 0 to 6;
or
—O—P(X)OH—O—[(CH₂)_{d}—C(O)—NH]_{b}—CHR—[C(O)—NH—(CH₂)ₑ]_{c}—O—P(X)OH—O— (II)
with b and c being independently an integer from 0 to 4, and the sum b+c is from 3 to 7;
d and e being independently an integer from 1 to 3; and
with R being -L_{f}-J,
X being O or S, L being a linker and f being an integer being 0 or 1, and J being a molecule facilitating the endocytosis or being H.

27. The conjugated nucleic acid molecule according to claim 26, wherein the nucleic acid molecule comprises one of the following sequences:
(SEQ ID NO 1)
5′CCCAGCAAACAAGCCT-∫
3′GGGTCGTTTGTTCGGA-∫
and
(SEQ ID NO 2)
5′CAGCAACAAG-∫
3′GTCGTTGTTC-∫
or a sequence wherein 1 to 3 nucleotides are substituted by a ribonucleotide or a modified deoxyribonucleotide or ribonucleotide.

28. The conjugated nucleic acid molecule according to claim 26, wherein the molecule facilitating the endocytosis is selected from the group consisting of a cholesterol, single or double chain fatty acids, ligand which targets a cell receptor enabling receptor mediated endocytosis, or a transferrin.

29. The conjugated nucleic acid molecule according to claim 26, wherein the molecule facilitating the endocytosis is a cholesterol.

30. The conjugated nucleic acid molecule according to claim 26, wherein the molecule facilitating the endocytosis is a ligand of a sigma-2 receptor (σ2R).

31. The conjugated nucleic acid molecule according to claim 30, wherein the ligand of a sigma-2 receptor (σ2R) comprises the following formula: 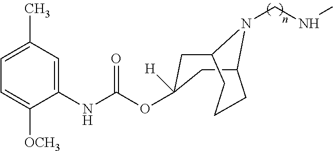
with n being an integer from 1 to 20.

32. The conjugated nucleic acid molecule according to claim 26, wherein 1, 2 or 3 internucleotidic linkages of the nucleotides located at the free end of the double-stranded moiety of the nucleic acid molecule have a modified phosphodiester backbone, optionally on both strands.

33. The conjugated nucleic acid molecule according to claim 26, wherein the loop has the formula (I) and K is

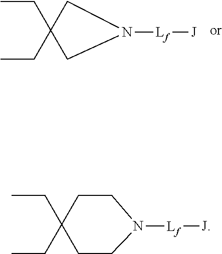

34. The conjugated nucleic acid molecule according to claim 26, wherein f is 1 and L is —C(O)—(CH₂)ₘ—NH—[(CH₂)₂—O]ₙ—(CH₂)ₚ—C(O)-J or —C(O)—(CH₂)ₘ—NH—[C(O)—CH₂—O]ₜ—[(CH₂)₂—O]ₙ—(CH₂)ₚ—[C(O)]ᵥ-J with m being an integer from 0 to 10; n being an integer from 0 to 6; and p being an integer from 0 to 2; t and v being an integer 0 or 1 with at least one among t and v being 1.

35. The conjugated nucleic acid molecule according to claim 26, wherein f is 1 and L-J is selected in the group consisting of —C(O)—(CH₂)ₘ—NH—[(CH₂)₂—O]ₙ—(CH₂)ₚ—C(O)-J, —C(O)—(CH₂)ₘ—NH—C(O)—[(CH₂)₂—O]ₙ—(CH₂)ₚ-J, —C(O)—(CH₂)ₘ—NH—C(O)—CH₂—O—[(CH₂)₂—O]ₙ—(CH₂)ₚ-J, —C(O)—(CH₂)ₘ—NH—C(O)—[(CH₂)₂—O]ₙ—(CH₂)ₚ—C(O)-J and —C(O)—(CH₂)ₘ—NH—C(O)—CH₂—O—[(CH₂)₂—O]ₙ—(CH₂)ₚ—C(O)-J, with m being an integer from 0 to 10; n being an integer from 0 to 15; and p being an integer from 0 to 3.

36. The conjugated nucleic acid molecule according to claim 26, wherein the loop has the formula (I)
—O—P(X)OH—O—{[(CH₂)₂—O]_{g}—P(X)OH—O}ᵣ—K—O—P(X)OH—O—{[(CH₂)₂—O]ₕ—P(X)OH—O—}ₛ (I)
with X being S, r being 1, g being 6, s being 0, and K being 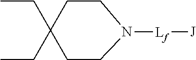
with f being 1 and L being C(O)—(CH₂)₅—NH—[(CH₂)₂—O]₃—(CH₂)₂—C(O)-J, —C(O)—(CH₂)₅—NH—C(O)—[(CH₂)₂—O]₃—(CH₂)₃-J, —C(O)—(CH₂)₅—NH—C(O)—CH₂—O—[(CH₂)₂—O]₅—CH₂—C(O)-J, —C(O)—(CH₂)₅—NH—C(O)—CH₂—O—[(CH₂)₂—O]₉—CH₂—C(O)-J, —C(O)—(CH₂)₅—NH—C(O)—CH₂—O— [(CH₂)₂—O]₁₃—CH₂—C(O)-J, or —C(O)—(CH₂)₅—NH—C(O)-J.

37. The conjugated nucleic acid molecule according to claim 26, wherein the conjugated nucleic acid molecule is selected from the group consisting of 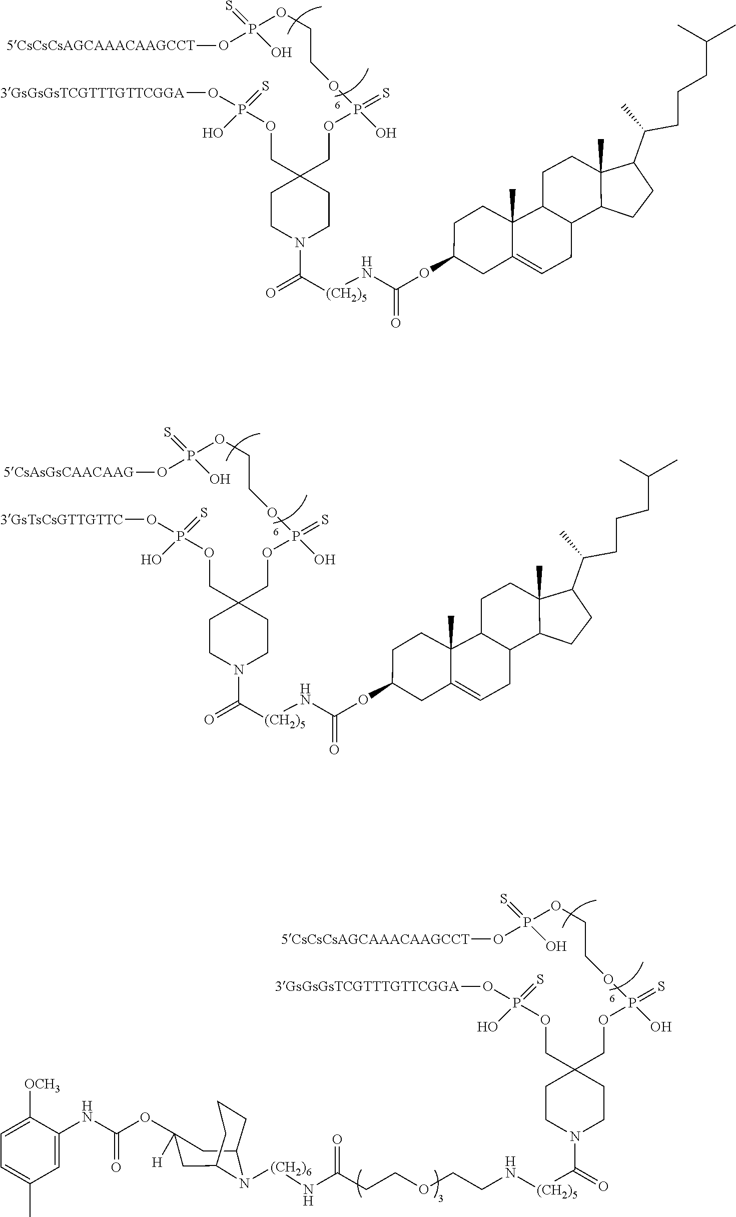 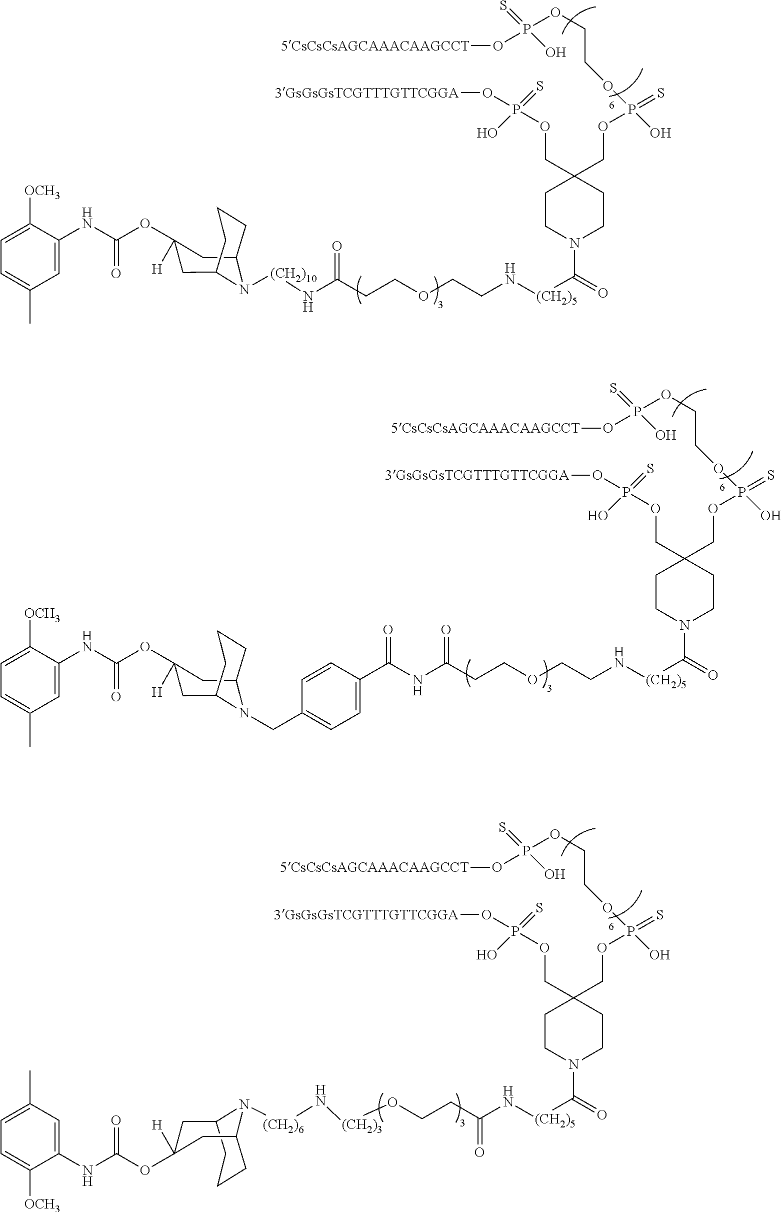 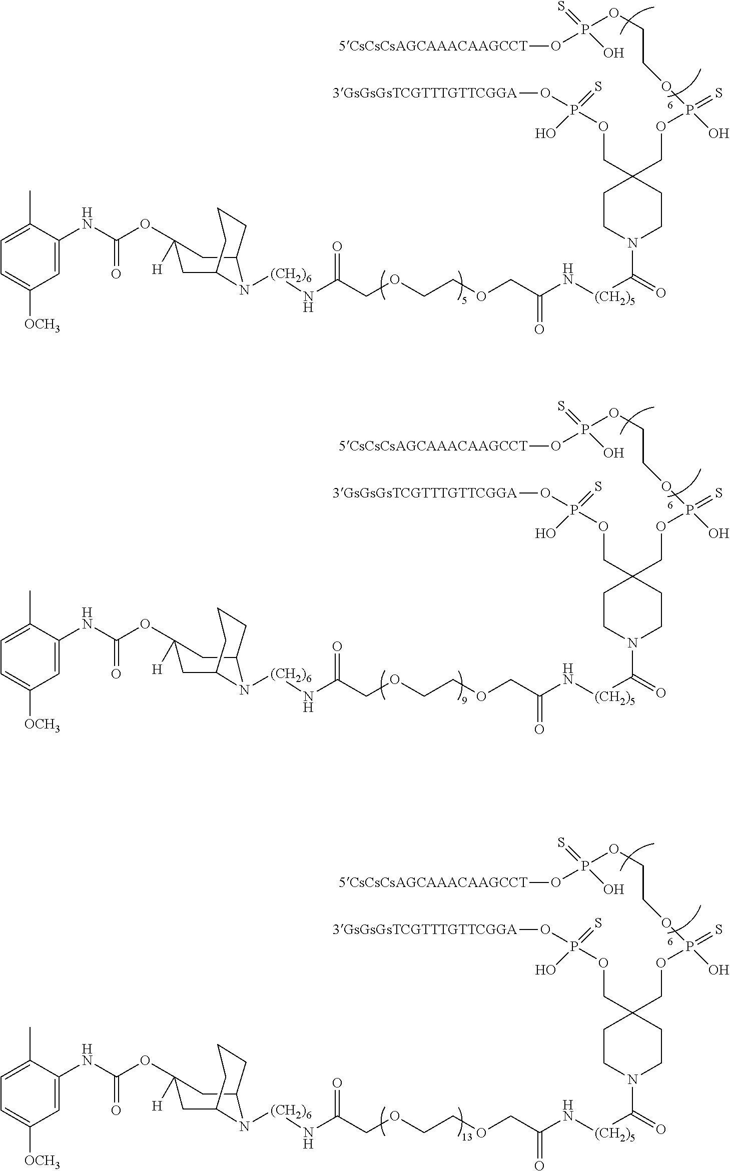 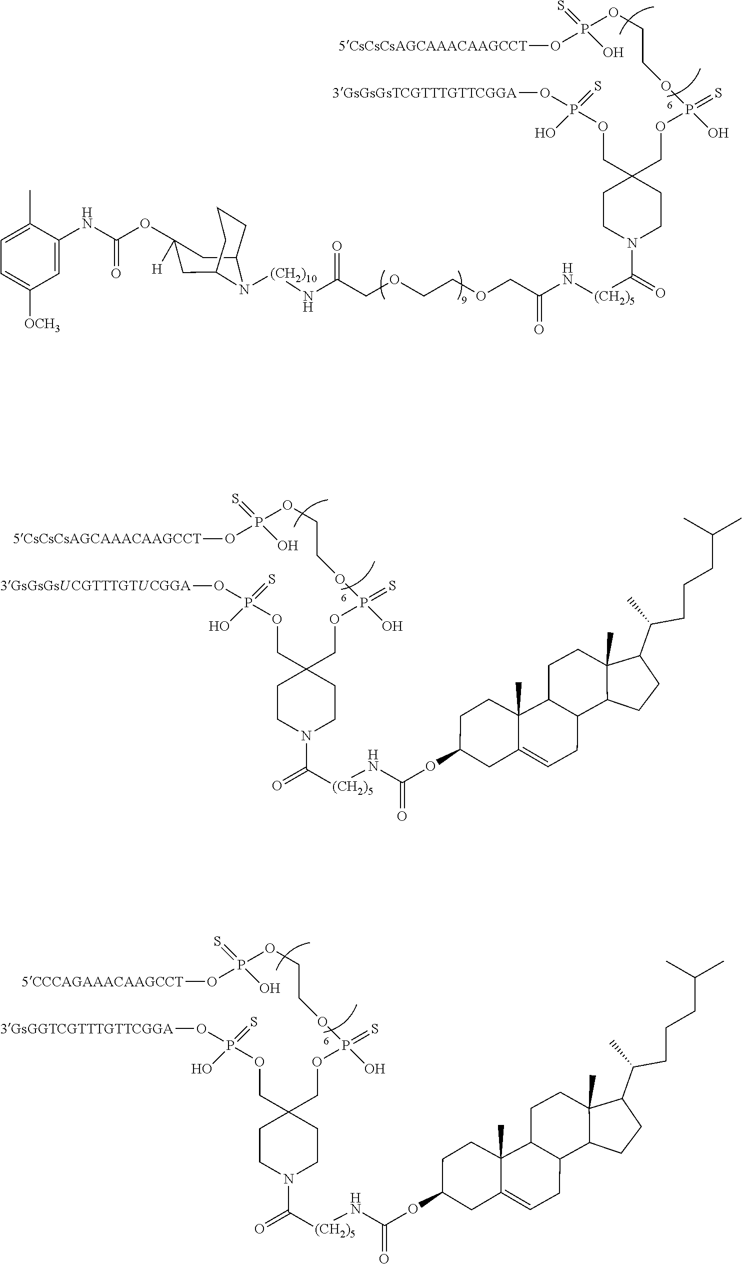 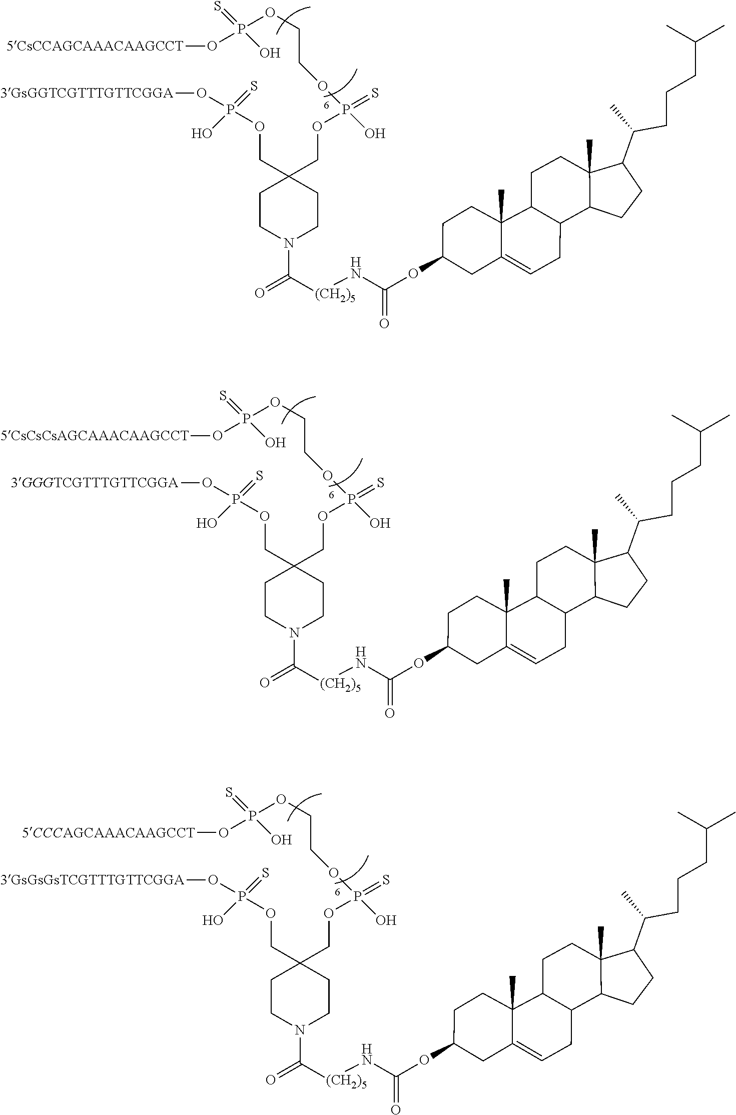
wherein internucleotide linkages “s” refers to phosphorothioate internucleotide linkages; italic U being 2′-deoxy-2′-fluoroarabinouridine, italic G being 2′-deoxy-2′-fluoroarabinoguanosine; italic C being 2′-deoxy-2′-fluoroarabinocytidine; and the pharmaceutically acceptable salts thereof.

38. The conjugated nucleic acid molecule according to claim 37, wherein the conjugated nucleic acid molecule is selected from the group consisting of 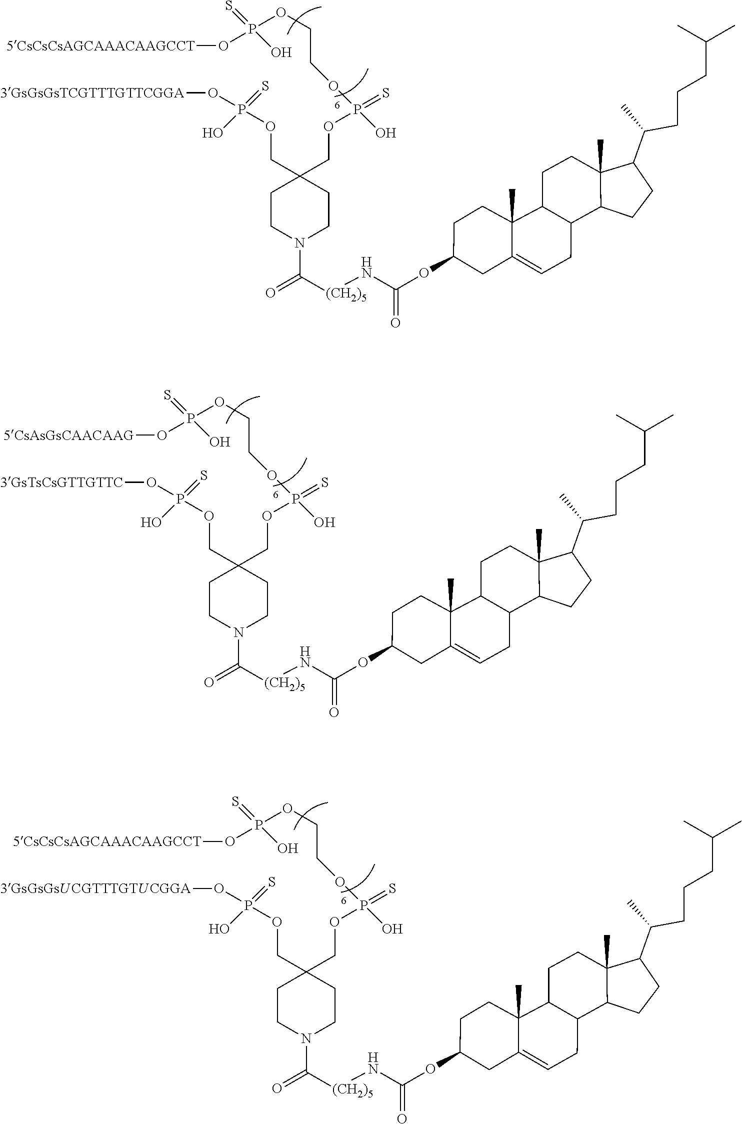 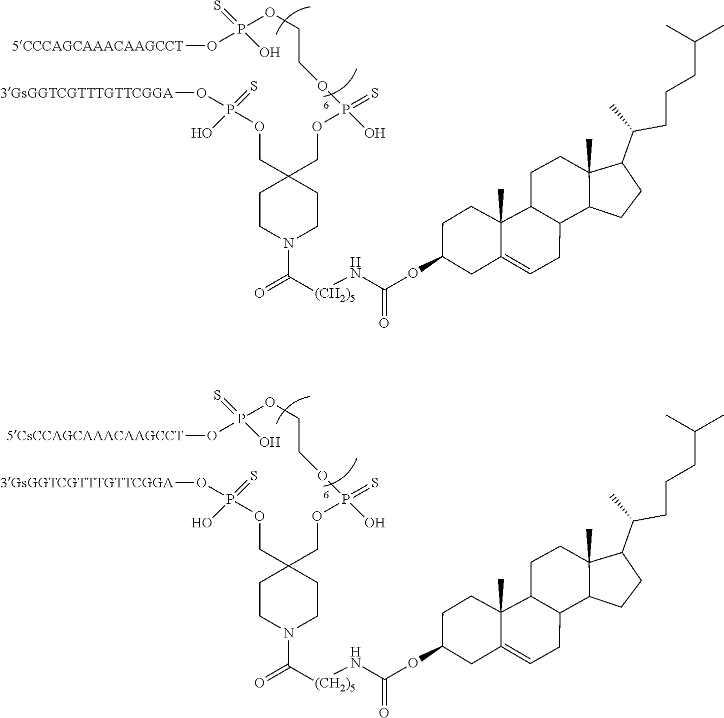 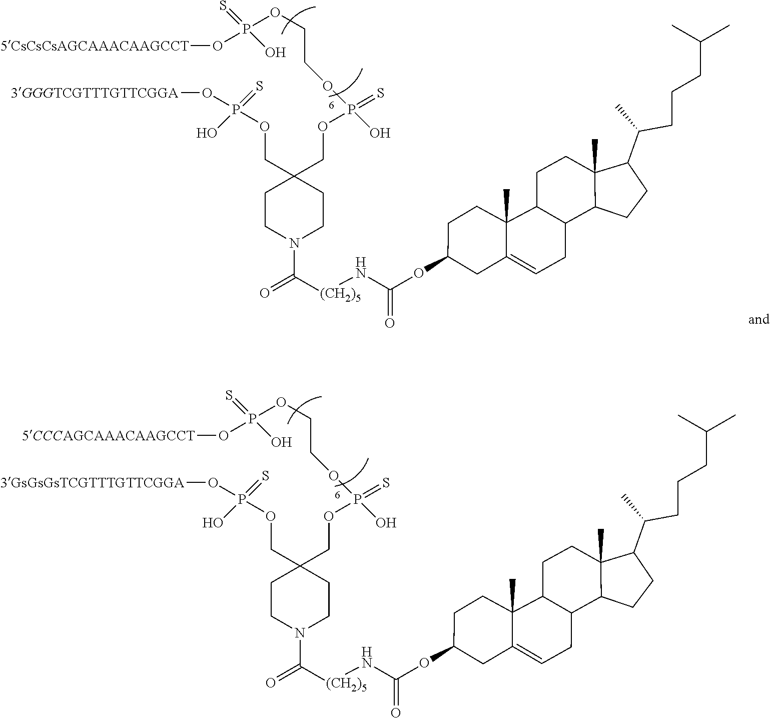
wherein internucleotide linkages “s” refers to phosphorothioate internucleotide linkages; italic U being 2′-deoxy-2′-fluoroarabinouridine, italic G being 2′-deoxy-2′-fluoroarabinoguanosine; italic C being 2′-deoxy-2′-fluoroarabinocytidine; or the pharmaceutically acceptable salts thereof.

39. The conjugated nucleic acid molecule according to claim 26, wherein the conjugated nucleic acid molecule is 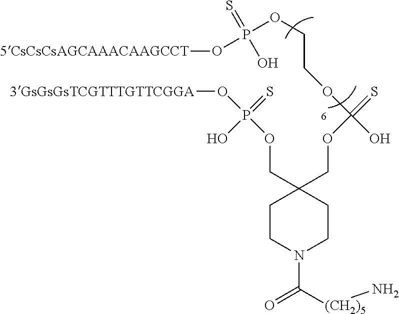
wherein internucleotide linkages “s” refers to phosphorothioate internucleotide linkages; or the pharmaceutically acceptable salts thereof.

40. A pharmaceutical composition comprising a conjugated nucleic acid molecule according to claim 26.

41. The pharmaceutical composition according to claim 40, wherein the pharmaceutical composition further comprises an additional therapeutic agent selected from an immunomodulator, an immune checkpoint inhibitor (ICI), a T-cell-based cancer immunotherapy, adoptive cell transfer (ACT), genetically modified T-cells or engineered T-cells, chimeric antigen receptor cells (CAR-T cells), a conventional chemotherapeutic, radiotherapeutic or anti-angiogenic agent, HDAC inhibitor, or targeted immunotoxin.

42. A method of treating a cancer in a subject in need thereof, comprising administering a therapeutically efficient amount of a conjugated nucleic acid molecule according to claim 26 or a pharmaceutical composition comprising said molecule repeatedly or chronically.

43. The method of treating a cancer according to claim 42, comprising administering repeated cycles of treatment.

44. The method of treating a cancer according to claim 42, wherein the patients have tumors carrying deficiencies in the NAD⁺ synthesis.

45. The method of treating a cancer according to claim 44, wherein the tumor cells further carry DNA repair pathways deficiencies selected from ERCC1 or ATM deficiency or IDHs mutations.

46. A pharmaceutical composition comprising a conjugated nucleic acid molecule according to claim 38.

47. A pharmaceutical composition comprising a conjugated nucleic acid molecule according to claim 39.

48. A method of treating a cancer in a subject in need thereof, comprising administering a therapeutically efficient amount of a conjugated nucleic acid molecule according to claim 38 or a pharmaceutical composition comprising said molecule repeatedly or chronically.

49. A method of treating a cancer in a subject in need thereof, comprising administering a therapeutically efficient amount of a conjugated nucleic acid molecule according to claim 39 or a pharmaceutical composition comprising said molecule repeatedly or chronically.
